(19) United States
(12) Patent Application Publication
(10) Pub. No.: US 2020/0397402 A1
(43) Pub. Date: Dec. 24, 2020

(54) ULTRASONIC METHODS FOR TRACKING STRESS FRACTURES OF BONE

(71) Applicant: Massachusetts Institute of Technology, Cambridge, MA (US)

(72) Inventors: Jonathan Richardson, Brookline, MA (US); Mitesh Amin, Rochester, NY (US)

(73) Assignee: Massachusetts Institute of Technology, Cambridge, MA (US)

(21) Appl. No.: 16/841,441

(22) Filed: Apr. 6, 2020

Related U.S. Application Data
(60) Provisional application No. 62/858,742, filed on Jun. 7, 2019.

(51) Int. Cl. A61B 8/08; A61B 8/00

(52) U.S. Cl. CPC A61B 8/0875; A61B 8/4494; A61B 8/0833; A61B 8/5215; A61B 8/461

(57) ABSTRACT

Devices and methods are provided herein for diagnosing, ranking, and/or tracking of stress fractures in biological materials such as bone and/or other hard tissue(s) using ultrasound imaging. The methods and devices provided herein include, for example: methods and devices for identifying a location on a bone; methods and devices for automated alignment of the transducer over a location of interest; and methods and devices for enhancing the sensitivity to in ultrasound imaging. The methods and devices provided herein may further provide for improved diagnosis, ranking, and/or tracking of stress fractures in bone and/or other hard tissue(s) by using signature characteristics of stress fractures.

### CROSS-REFERENCE TO RELATED APPLICATIONS

This application claims the benefit under 35 U.S.C. § 119(e) of U.S. provisional patent application Ser. No. 62/858,742, entitled “ULTRASONIC METHODS FOR TRACKING STRESS FRACTURES OF BONE”, filed Jun. 7, 2019 under Attorney Docket No. M0437.70149US00, which is hereby incorporated by reference in its entirety.

### STATEMENT REGARDING FEDERALLY SPONSORED RESEARCH

This invention was made with Government support under Grant No. FA8702-15-D-0001 awarded by the U.S. Air Force. The Government has certain rights in the invention.

### TECHNICAL FIELD

Devices and methods for using ultrasound to evaluate stress fractures in biological materials such as bone and/or other hard tissue(s) are provided herein. These devices and methods are useful for diagnosing, ranking, and/or tracking stress fractures in biological materials such as bone and/or other hard tissue(s) in patients.

### BACKGROUND

Stress fractures and/or stress responses in bones often occur when individuals are subject to repeated physical stress, as is typical for athletes or military recruits during rigorous training, for example. Stress fractures of the foot and ankle alone represent up to 20% of sports medicine clinical visits, while stress fractures elsewhere in the body are also common.

Existing methods for diagnosing stress fractures in bones include imaging by x-ray, nuclear scintigraphy, and magnetic resonance imaging (MRI). Treatment for stress fractures includes cessation of strenuous activity (e.g., no running, dancing, etc.) and, in some cases, cessation of all weight bearing activity on the affected limb for up to three months or more. Such treatment results in an interruption to daily life for professional athletes, but is also very burdensome for the general public (including amateur athletes and dancers, for example).

### SUMMARY

Some embodiments provide for a method for examining bone and/or hard tissue of a patient using ultrasound imaging, the method comprising: transmitting one or more ultrasound waves to a location of interest; receiving one or more ultrasound waves from the location of interest, the one or more received ultrasound waves comprising reflections of at least some of the one or more transmitted ultrasound waves; determining an amount of received ultrasound waves comprising specular reflection and an amount of received ultrasound waves comprising diffuse reflection; and using the determined amounts of specular reflection and diffuse reflection to determine if the location of interest contains a defect.

Some embodiments provide for a method, comprising: using at least one of specular reflection of ultrasound waves from a location of interest or diffuse reflection of ultrasound waves from the location of interest to determine information about bone and/or other hard tissue comprising at least a portion of the location of interest.

Some embodiments provide for a method for positioning an ultrasound examination device relative to a location of interest in a patient, the method comprising: performing an ultrasound scan at a region including at least the location of interest; determining a plurality of scan positions in which a received signal of the ultrasound scan is maximized; and defining a scan trajectory comprising the plurality of scan positions where the received signal of the ultrasound scan is maximized.

### BRIEF DESCRIPTION OF THE DRAWINGS

Various non-limiting embodiments of the technology are described herein with reference to the following figures. It should be appreciated that the figures are not necessarily drawn to scale. Items appearing in multiple figures are indicated by the same reference numeral in all figures in which they appear. For purposes of clarity, not every component may be labeled in every drawing.

FIG. 1 illustrates example images from different techniques for imaging stress fractures in bone and/or other hard tissue(s), in accordance with some embodiments of the technology described herein.

FIG. 2 illustrates an example embodiment of an automated stress fracture diagnosis, ranking, and tracking apparatus, in accordance with some embodiments of the technology described herein.

FIG. 3 illustrates example elastic wave scatter simulations from a bone surface, in accordance with some embodiments of the technology described herein.

FIG. 4 illustrates example graphs depicting the “scatter matrix” from an ABS tube, in accordance with some embodiments of the technology described herein.

FIG. 5 illustrates an example embodiment of an automated stress fracture diagnosis, ranking, and tracking apparatus, in accordance with some embodiments of the technology described herein.

FIGS. 6A-6D illustrate example methods for diagnosing, ranking, and tracking stress fractures in bones and/or other hard tissue(s), in accordance with some embodiments of the technology described herein.

FIG. 7 illustrates an example graph illustrating the difference in receive ultrasound signal between damaged bone and undamaged bone, in accordance with some embodiments of the technology described herein.

FIG. 8 illustrates a series of images depicting the frequency content of scatter from a lesion on the bone surface, in accordance with some embodiments of the technology described herein.

FIG. 9 illustrates example ultrasound images showing specular and diffuse reflection in damaged and undamaged ABS pipe, in accordance with some embodiments of the technology described herein.

FIG. 10A illustrates an example experimental setup for measuring specular and diffuse reflection in a damaged sheep bone, in accordance with some embodiments of the technology described herein.

FIG. 10B illustrates example ultrasound images showing specular and diffuse reflection in damaged and undamaged sheep bone, in accordance with some embodiments of the technology described herein.

FIG. 11A illustrates example ultrasound images showing specular and diffuse reflection in damaged and undamaged sheep bone, in accordance with some embodiments of the technology described herein.

FIG. 11B illustrates an example difference image between ultrasound images of damaged and undamaged sheep bone, in accordance with some embodiments of the technology described herein.

FIGS. 12-20 are a series of example graphs and illustrations depicting the use of the devices and methods provided herein in various studies to perform measurements with phantoms, e.g., plastic bone simulants, excised bare animal bones (i.e., with the tissue(s) removed), and excised intact animal limbs (i.e., with the tissue(s) intact), in accordance with some embodiments of the technology described herein.

FIG. 21 is a block diagram of a general purpose computing machine, in accordance with some embodiments of the technology described herein.

### DETAILED DESCRIPTION

Provided herein are devices and methods for using ultrasound to evaluate stress fractures in biological materials such as bone and/or other hard tissue(s). These devices and methods are useful in a number of applications, such as, for example, diagnosing the presence of stress fractures in bones and/or other hard tissue(s), particularly below the knee; ranking severity of stress fractures in bone and/or hard tissue(s), particularly below the knee; tracking recovery from stress fractures of bone and/or other hard tissue(s), particularly below the knee; and combinations thereof. These devices and methods are useful in cases where more expensive methods (e.g., MRI, scintigraphy) are not available, such as in a smaller clinical setting.

Stress fractures are tiny cracks in bone often caused by repeated force or repeated physical stress, such by overuse of weight bearing bones (e.g., in the lower leg and foot) from running, jumping, walking, or other activities. Stress fractures may also arise from normal use of a bone which is in a weakened condition (e.g., from osteoporosis). Pain resulting from a stress fracture may be gradual at first, but may increase over time, particularly if subjected to weight bearing activity. In addition to extended recovery times in which an individual must reduce or cease all weight bearing activity on the afflicted area, certain “high risk” stress fractures may worsen over time, including leading to a complete break in a bone, if not properly treated. Thus, proper diagnosis, ranking, and tracking of stress fractures in bone and/or other hard tissue(s) are significant factors in improving an individual's recovery.

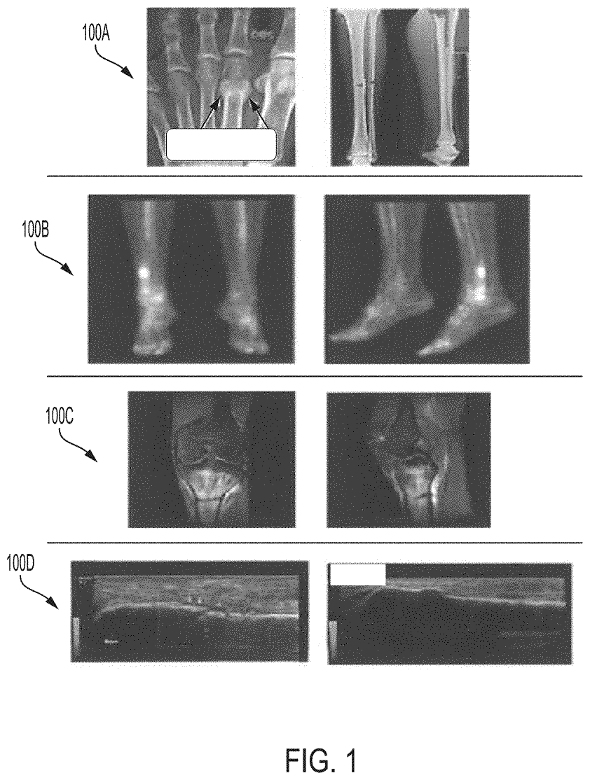

In current treatment methods, a stress fracture would be diagnosed on the basis of localized pain, followed by imaging, for example, by x-ray, nuclear scintigraphy, and/or magnetic resonance imaging (MRI). FIG. 1 illustrates example images from these different imaging techniques. In particular, images 100A are examples of images acquired using x-rays, images 100B are examples of images acquired using nuclear scintigraphy, and images 100C are examples of images acquired using MRI. The inventors have recognized, however, that while existing imaging methods offer certain advantages, they also suffer from significant drawbacks which limit their efficacy and availability as appropriate imaging modalities in this context. Standard x-ray offers poor image resolution making it difficult to detect small features of a stress fracture and to track stress fractures over time. Nuclear scintigraphy involves significant whole-body radiation dosing and the scanning process can be hours to days long making repeated scans unlikely. MRI is thought to offer the best diagnostic of the existing imaging techniques. However, MRI is complex, expensive, and difficult to offer outside of a major hospital setting. In particular, MRI systems are typically large, heavy, consume large amounts of power, and may require dedicated shielded facilities. Thus, the inventors have recognized there is a need for improved (e.g., more accessible, accurate and efficient) devices and techniques for diagnosing stress fractures in biological materials such as bone and/or other hard tissue(s).

As described herein, treatment for stress fractures include cessation of physical activity for an extended period of time (e.g., three months, or more, in some cases). Such treatment may constitute a significant interruption to a patient's daily life which lasts until the stress fracture is healed. Improved ranking, for example, of the severity of stress fractures, and tracking, for example, of the recovery of the stress fracture, may provide patients with more precise information on a recovery timeline, including when it is safe to return to normal activities.

The inventors have recognized that devices and techniques using ultrasound to image a patient provides for improved diagnosis, ranking, and tracking of stress fractures in patients. In particular, the devices and methods described herein using ultrasound exhibit a number of advantages over current therapies and methods (e.g., x-rays, nuclear scintigraphy, MRI). For example, using ultrasound to diagnose stress fractures is much more cost effective than existing methods. Ultrasound is currently being miniaturized by several companies, lowering costs further. The ultrasound equipment market generates $6.6B per year with multiple vendors. Ultrasound imaging may also be implemented using a portable probe and/or system, making it easier to deploy and increasing its accessibility. Unlike existing imaging modalities, ultrasound does not use radiation, allowing for repeatable scans of patient anatomy which are relatively short in duration. In some embodiments, the devices and methods provided herein are added to or otherwise used in conjunction with available commercial equipment, expanding its utility to hard tissue problems.

The devices and methods provided herein provide an inexpensive and noninvasive method of diagnosing, ranking, and/or tracking stress. The devices and methods provided herein use ultrasonic methods with improved sensitivity to the signatures of stress fractures. The methods and devices provided herein include, for example: methods and devices for identifying a location on a bone; methods and devices for automated alignment of the transducer over a location of interest; methods and devices for enhancing sensitivity in ultrasound imaging; and using signature characteristics of stress fractures.

While the examples provided herein demonstrate use in evaluating stress fractures in bone, particularly those below the knee, it is understood that the devices and methods provided herein are also useful in diagnosing stress fractures in other parts of the body. Furthermore, the devices and methods described herein may be applied in other contexts, for example, other contexts which include imaging of bone surface.

(1) Device Overview

The devices and methods described herein provide for using ultrasound imaging to diagnose, rank, and track stress fractures in bone and/or other hard tissue. Ultrasound is a noninvasive imaging modality which utilizes high-frequency sound waves to image a patient. Ultrasound systems typically include an examination device (e.g., a probe) comprising a plurality of ultrasonic transducers configured to transmit an ultrasound wave (e.g., a pulse) towards a location of interest. Transmit waves are reflected off structures in the body and received by an examination device. The received waves are processed to produce an ultrasound image of the location of interest. Typically, the examination device is configured to both transmit and receive ultrasound pulses with one or more transducers, for example, with the same set of transducers to perform transmit and receive functions or with separate sets of transmit and receive transducers on the same probe. In other embodiments, transmit and receive functions may be performed by one or more separate examination devices.

Ultrasound as an imaging modality is useful as a more accessible alternative to more expensive techniques, such as MRI. However, existing ultrasound systems are configured to image soft tissue and are not configured to study bone and/or other hard tissue(s). Stress fractures are most problematic when they involve an outer bone surface, such as the cortical bone. Thus, the inventors have recognized that in order to apply ultrasound imaging to diagnosis, ranking, and tracking of stress fractures, devices and techniques that are capable of producing ultrasound images of bone and/or other hard tissue(s) must be developed.

Stress fractures exhibit certain characteristics during the recovery process which may be used to better diagnose, rank, and/or track stress fractures using ultrasound techniques. For example, stress fractures begin with a stress response in which edema within bone marrow causes increased pressure and increased diffusion of fluid through the bone structure, creating what is known as a “bone bruise” which may be exhibited as pitting on the surface of the bone. The inflammation caused by the stress response causes rapid remodeling within the bone at the bone surface, and the bone is weakened due to remodeling. Larger fractures, which may be detectable through imaging, occurs at points where the bone is weakened. During recovery, a callus forms on the bone surface and subsides after full recovery. In addition, the microcracking at the bone surface leading to the stress fracture may be detected through ultrasound imaging methods having sufficient sensitivity to detect sub-wavelength scale features.

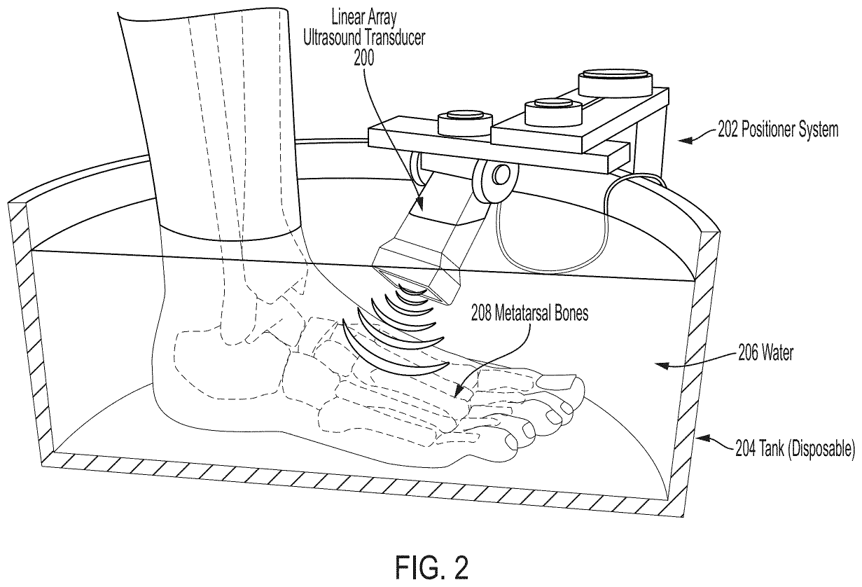

To study the effects of stress fractures in bone and/or other hard tissue(s), the inventors have developed an ultrasound imaging device for imaging bone and/or other hard tissue(s) at a location of interest (e.g., at the location of a suspected stress fracture). FIG. 2 illustrates an example embodiment of an automated stress fracture diagnosis, ranking, and tracking apparatus, in accordance with some embodiments of the technology described herein. The apparatus 200 may be used to automatically position an ultrasound examination device 201 of the apparatus 200 to image a location of interest by scanning the location of interest with one or more ultrasound waves and processing signals of received waves reflected from the location of interest. As described herein, the inventors have developed methods for acquiring and processing ultrasound signals to enhance sensitivity of the ultrasound examination device, for example, to subtle sub-wavelength features in bone and/or other hard tissue(s). In the illustrated embodiment, the apparatus is configured to image the metatarsal bones 208 of a foot. Stress fractures occurring in the foot and ankle account for about 35% of stress fractures in bones, with about 75% of stress fractures occurring below the knee. The devices and techniques described herein provide for improved diagnosing, ranking, and tracking of stress fractures, particularly for stress fractures occurring below the knee.

The apparatus 200 comprises an ultrasound examination device 201 (e.g., a probe, in some embodiments). The ultrasound examination device 201 may comprise an array of ultrasound transducer elements configured to transmit and/or receive ultrasound waves to/from the location of interest. Any suitable type of ultrasound transducer elements may be used, for example, micromachined ultrasound transducers, such as a capacitive micromachined transducers (CMUT) and/or piezoelectric micromachined ultrasonic transducers (PMUT). The ultrasound transducer elements may be arranged as a linear array. Linear array transducers are advantageous as they tend to produce the best overall image quality relative to other types of transducer arrays, however, in some embodiments, the ultrasound transducer elements may be arranged in different configurations other than that shown in FIG. 2.

As described herein, the ultrasound examination device 201 may comprise ultrasound transducer elements configured to transmit and/or receive ultrasound waves to/from the location of interest, referred to generally herein as transmit transducers and receive transducers. In some embodiments, such as in the illustrated embodiment, the transmit and receive transducers may be disposed on a same substrate. In some embodiments, one or more (e.g., all) of the ultrasound transducer elements may be configured to interchange between performing the transmit function and the receive function. In other embodiments, the transmit and receive transducers may be separate elements with at least some transducers performing the transmit function, and at least some transducers performing the receive function. In some embodiments at least some of the ultrasound transducer elements performing the transmit function and at least some of the ultrasound transducer elements performing the receive function may be disposed on separate substrates. For example, as described herein, the ultrasound examination device may comprise multiple transmit and/or receive probes disposed at different angles to maximize capture of specular and diffuse reflection from a location of interest.

The ultrasound transducer elements may be configured to perform the transmit function using a “synthetic aperture” (SA) approach. The inventors have recognized that it may be advantageous to measure the specular and diffuse scatter from a location of interest, as described herein. However, standard ultrasound imaging does not allow for evaluating diffuse vs. specular scatter. In fact, some types of ultrasound imaging (e.g., B-mode imaging) essentially measures backscatter only. Thus, in some embodiments, a SA ultrasound imaging approach is applied herein to acquire both specular and diffuse reflection data. Once the ultrasound examination device 201 is aligned with the bone surface, each ultrasound transducer element is energized individually in turn while all ultrasound transducer elements are monitored for receive signals. The received signals from the linear transducer array may be combined in post-processing rather than as the image data is acquired. Doing so allows for the received data to be exploited in a more flexible manner, higher lateral resolution images, and deeper penetration of the transmit ultrasound waves. For example, focal points of the transmit and receive waves may be selected after-the-fact of image acquisition, allowing for focusing on bone and eliminating interference from tissue surrounding the bone. The acquired data set may allow for studying backscatter or oblique scatter (e.g., including both specular scatter and diffuse scatter). As described herein, the inventors have recognized that it is advantageous to apply the SA approach in stress fracture imaging as it enables capture of scatter patterns from the irregular surface of the bone (caused by pitting and/or microcracks in the bone) while simplifying the positioning and alignment of the ultrasound examination device itself.

The apparatus 200 may further comprise a positioner system 202 (e.g., a robotic arm) to facilitate automatic positioning of the ultrasound examination device 201. As described herein, automated positioning may be used to acquire an image of the overall shape of patient anatomy containing the location of interest to create a bone contour map to facilitate positioning the ultrasound examination device 201 in subsequent scans. The positioner system 202 may perform a sweeping scan of the overall shape of the patient anatomy by moving the ultrasound examination device about a base, for example, in three-dimensions, to which the ultrasound examination device 201 and positioner system 202 may be coupled. In the illustrated embodiment, the base comprises a tank 204, as described further herein, for holding the patient anatomy. The positioner system 202 may also be configured to precisely align the ultrasound examination device 201 and the ultrasound transducer elements with the location of interest by making small movements of the ultrasound examination device 201 about the base and tilting the ultrasound examination device 201 to a desired angle with respect to the patient anatomy and location of interest.

The positioner system 202 may be combined with the techniques described herein for positioning the ultrasound examination device at a location of interest based on the received ultrasound signal itself (e.g., by adjusting the ultrasound examination device to maximize the signal received from the location of interest) As described herein, ultrasound has not previously been used as an imaging technique for diagnosing, ranking, and evaluating stress fractures in bone and/or other hard tissue as ultrasound imaging is traditionally used to provide images of soft tissue, while stress fractures occur in bone, particularly at the outer surface of the bone (e.g., at the cortical bone). Imaging of the bone surface requires careful alignment of the ultrasound examination device such that it captures the reflection of sound from the bone surface and not merely from soft tissue surrounding the bone. The rapid remodeling and pitting at the bone surface caused by a stress fracture and preceding stress response may make alignment more difficult. Thus, automated positioning of the ultrasound examination device 201 using the positioner system 202 and alignment techniques described herein may facilitate the careful positioning and alignment of the ultrasound examination device relative to the location of interest such that imaging of the bone surface is possible.

Although the devices and techniques described herein are described with reference to an automated positioner system and to automated positioning and alignment of an ultrasound examination device, in some embodiments, scanning of the patient anatomy containing the location of interest and positioning of the ultrasound examination device relative to the location of interest may be performed manually.

In some embodiments, the apparatus further comprises a tank 204 containing a liquid 206 such as water. In order to facilitate transmission of the transmit ultrasound waves to the location of interest, it is advantageous to use a conductive medium that creates a bond between the ultrasound transducer elements and the skin, as air alone typically does not provide a sufficient medium for the ultrasound waves to travel through. Traditionally, when performing ultrasound imaging, typically, a gel comprising water and propylene gycol is spread over the location of interest to serve as the conductive medium. In embodiments where the location of interest is not precisely known, it may be necessary to perform a sweeping scan of the patient anatomy to localize the potential stress fracture. The inventors have recognized that surrounding the patient anatomy with a coupling medium, for example, by using water in a tank with the patient anatomy inserted therein such that the water surrounding the patient anatomy serves as the coupling medium between the ultrasound examination device and the patient anatomy may simplify the positioning of the ultrasound examination device as it permits an arbitrary scanning range to be specified by the automated positioner system 202, thus allowing the ultrasound examination device 201 to “search” for the location of interest before more precisely aligning the ultrasound transducer elements relative to the location of interest. In other embodiments, however, the techniques described herein may be implemented using an ultrasound gel and/or a polymer pad to couple the ultrasound examination device to the patient.

An apparatus, such as the apparatus 200 shown in FIG. 2, may be used to study stress fractures in bone and/or other hard tissue(s) according to the techniques described further herein. Briefly, the ultrasound examination device 201 may be automatically positioned to image the metatarsal bones 208, for example, of a foot positioned within a water tank 204. The positioner system 202 may move the ultrasound examination device 201 such that it scans one or more bones of the foot to produce an image of the overall bone contour in the foot. Using the overall contour of the bone(s), the automated positioner system 202 orients the ultrasound examination device 201 to a location of interest (LOI). The LOI could be, for example: a suspected stress fracture; a new region as identified by the clinician or patient; and/or a region known from a prior visit. The ultrasound examination device 201 is then automatically adjusted to maximize the return signal from the LOI. Synthetic aperture (SA) data is acquired to evaluate the LOI. SA data is analyzed to 1) produce a high-fidelity map of the bone contour nearby and at the LOI, e.g., generally along the long axis of the bone in some embodiments, and 2) evaluate both the specular and diffuse scatter at and nearby the LOI. In some embodiments, the SA data is compared to a prior dataset, if available, to evaluate the condition of the stress fracture.

(2) Methods and Devices for Identifying a Location on a Bone

According to an aspect of the technology described herein, there are provided methods and devices for identifying a location on a bone, for example, a location on a bone that can be revisited reliably on subsequent ultrasound scans and/or for any other suitable purpose. According to an aspect of the technology described herein, there is a provided a technique for identifying a location of interest using overall bone contour and localized features, as measured using, for example, an automated scan of the bone using a linear-array ultrasound transducer probe in a transverse orientation. At the start of a subsequent scan, the bone contour can be re-acquired and analyzed to find the location of prior scans, ensuring that repeated scans can be overlaid with adequate accuracy to track changes over time. In some embodiments, such techniques may be facilitated using an automated positioning system, such as the positioner system 201 shown in FIG. 2.

As described herein, a method for diagnosing, ranking, and/or tracking a stress fracture in bone and/or other hard tissue(s) may begin by performing a sweeping scan of patient anatomy containing the location of interest. For example, the scan may include a set of transverse scans (slices) of the bone contour. The sweeping scan collects ultrasound data at and surrounding the location of interest such that a high-fidelity mapping of the bone contour at and around the location of interest (e.g., along the long axis of the bone) may be produced with the collected ultrasound data. For example, the acquired images from the set of transverse scans may be used to locate the bone contour in each image algorithmically. The bone contour may be used to locate a location of interest, as described herein.

For example, the bone contour mapping may be used to identify localized features of the ultrasound image which define the location of interest. The localized features may be used in subsequent scans to reposition the ultrasound examination device at the location of interest such that the location of interest can be repeatedly monitored through the stress fracture healing process. For example, at the start of a subsequent scan, the ultrasound examination device can perform a sweeping scan to recollect ultrasound data of the bone contour near and at the location of interest. The localized features identified in the prior scan may be identified in the subsequent scan to correlate the prior and subsequent scans. The initial image acquisition to define the bone contour may be facilitated by the water and tank shown in FIG. 1 which permits scanning over an arbitrary range of angles.

In some embodiments, the methods and devices described herein for identifying a location on a bone are used to identify a location on a bone that can be revisited during a subsequent ultrasound scan, for example. As described herein, the recovery period for stress fractures may span three months, on average. Tracking the healing of a stress fracture following an initial diagnosis is an important factor in providing patients with accurate recovery timelines. Resuming normal activity too early before the healing process has been completed may cause a stress fracture to worsen. Thus, accurate tracking of a stress fracture to indicate when individuals will be able to resume normal weight bearing activities is necessary. One aspect of accurate tracking of a stress fracture in bone and/or other hard tissue(s) is ensuring that subsequent imaging of the location of interest are correlated in terms of location within a particular bone. As such, the methods and devices for identifying a location on a bone may provide for improved tracking of a stress fracture in bone and/or other hard tissue(s).

In some embodiments, additionally or alternatively, the bone contour map may further facilitate analysis of subsequent scan images by ensuring that subsequent scans can be overlaid onto previously acquired images to track changes in the stress fracture over time. As described herein, accurate tracking of a stress fracture is a significant factor in a patient's recovery.

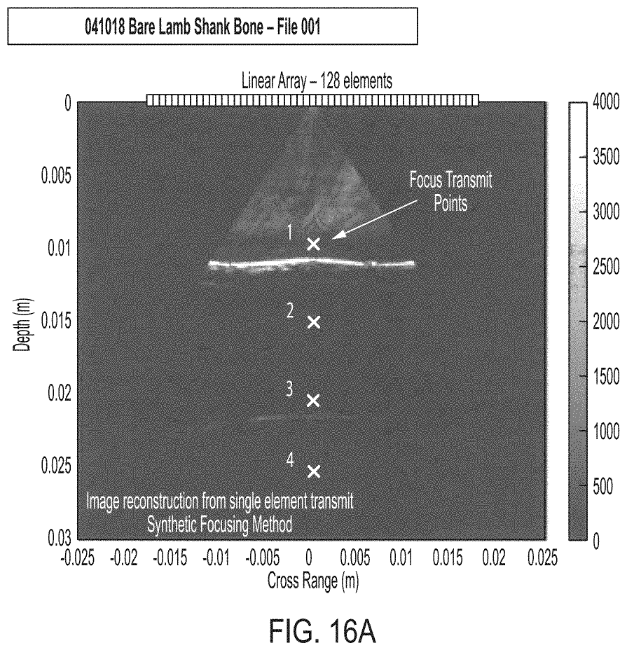

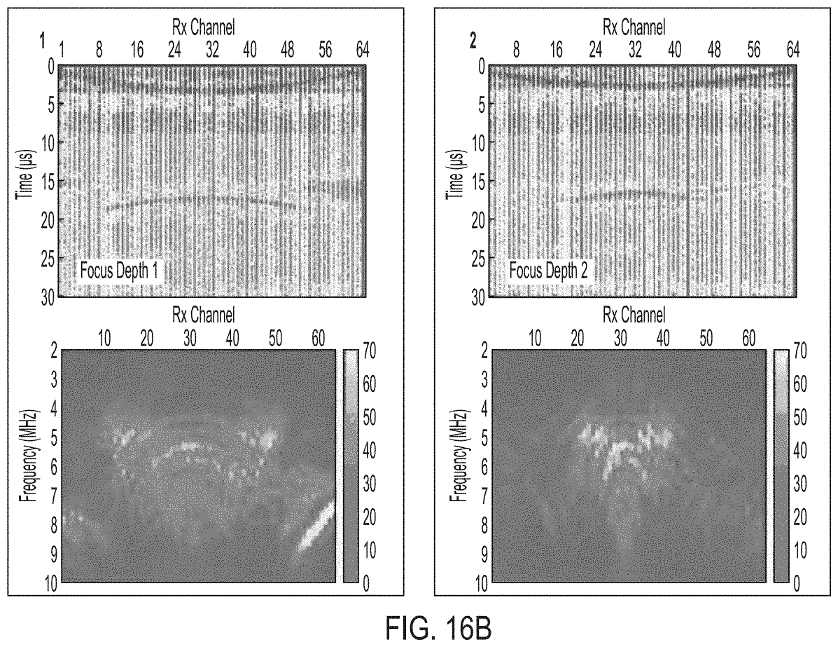

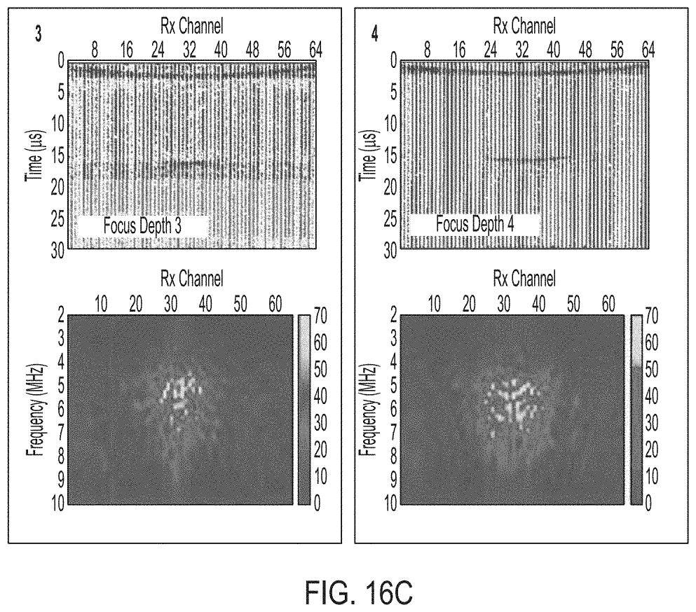

The inventors have implemented the concepts described herein, for example, for generating a bone contour map for identifying a location on bone (e.g., a location that can be revisited reliably on subsequent ultrasound scans). For example, FIGS. 16-18 and the accompanying description illustrate experimental results of the techniques described herein.

(3) Methods and Devices for Automated Alignment of the Transducer Over a Location of Interest

According to an aspect of the technology described herein, there are provided methods and devices for automated alignment of the ultrasound examination device and/or the ultrasound transducer elements over a location of interest. In particular, after performing the sweeping scan and determining the overall bone contour of the patient anatomy, bone contour may be used to move the ultrasound examination device to the location of interest and thereafter the ultrasound examination device may perform smaller adjustments in movement at or near the location of interest. The location of interest may be the location of a suspected stress fracture, a location identified by a clinician or the patient, and/or a location known from a prior visit, or other. In some embodiments, the techniques described herein for automated alignment include determining a trajectory along which the ultrasound examination device may be moved. The trajectory may be repeatable, for example, on subsequent scans performed by the ultrasound examination device and/or any other suitable device.

The ultrasound examination device may comprise one or more sensors (e.g., one or more inertial sensors or any other suitable sensor(s)) which facilitate location tracking of the ultrasound examination device. For example, data from the one or more sensors may be used to determine a plurality of positions defining the repeatable trajectory.

In some embodiments, the methods and devices provided herein for automated alignment of the ultrasound examination device use the collected ultrasound signal itself to align the ultrasound examination device over a location of interest. In particular, the collected ultrasound signal may be analyzed to determine one or more locations at which the collected ultrasound signal is maximized. The signal analysis may be performed during imaging and/or after a scan is completed.

For example, reflection from the location of interest is will be higher along the line of a bone, but there will be little to no reflection where there is no bone. Thus, one or more positions where reflection is maximized may be determined to determine a trajectory which moves along the bone contour. The automated alignment techniques described herein facilitate determination of a repeatable line scan trajectory, for example, including the one or more positions where reflection is maximized, in which the automated positioner system can move the ultrasound examination device to collect ultrasound signal from the location of interest. The scan trajectory may then be repeated to collect ultrasound data from the bone and/or other hard tissue at the location of interest to diagnose, rank, and/or track a suspected stress fracture. The scan trajectory may be stored in the form of executable code in memory of the ultrasound examination device and/or an external device coupled to the ultrasound examination device and/or the positioner system, such that the scan trajectory may be repeated any desired number of times.

As described herein, positioning of the ultrasound examination device, in some embodiments, is accomplished using robotic or otherwise automatic positioning equipment (e.g., the positioner system shown in FIG. 2). In other embodiments, positioning of the ultrasound examination device may be performed manually.

(4) Methods and Devices for Enhancing Sensitivity in Ultrasound Imaging

According to an aspect of the technology described herein, there is provided methods and devices for enhancing sensitivity in ultrasound imaging. For example, the methods and devices described herein may be used to enhance sensitivity of an ultrasound examination device to subtle sub-wavelength scale features, for example, in order to accurately diagnose, rank, and/or track stress fractures in bone and/or other hard tissue(s).

As described herein, stress fractures in bone and/or other hard tissue(s) may be exhibited by pitting, cracking, and/or other defects in the bone surface which are typically on the sub-wavelength scale (e.g., having a dimension less than the wavelength of the transmit wave, including less than or equal to 300 μm for ultrasound imaging). Existing techniques for imaging of stress fractures in bone and/or other hard tissue(s) may lack the sufficient resolution necessary to identify these features, and thus may fail to properly diagnose a stress fracture. Thus, according to an aspect of the technology described herein, there are provided methods and devices for enhancing the sensitivity to subtle sub-wavelength scale features of a stress fracture/stress response at a particular location.

In some embodiments, the devices and methods provided herein for enhancing sensitivity in ultrasound imaging are based on measuring both the direct, i.e., “specular”, and diffuse scatter from the bone surface and comparing magnitudes. Specular reflection is mirror-like reflection of waves from a surface wherein incident waves are reflected at the same angle to the surface normal as the incident ray on the opposing side of the surface normal. By contrast, diffuse reflection is reflection of waves from a surface such that the incident wave is scattered from the surface normal at many different angles.

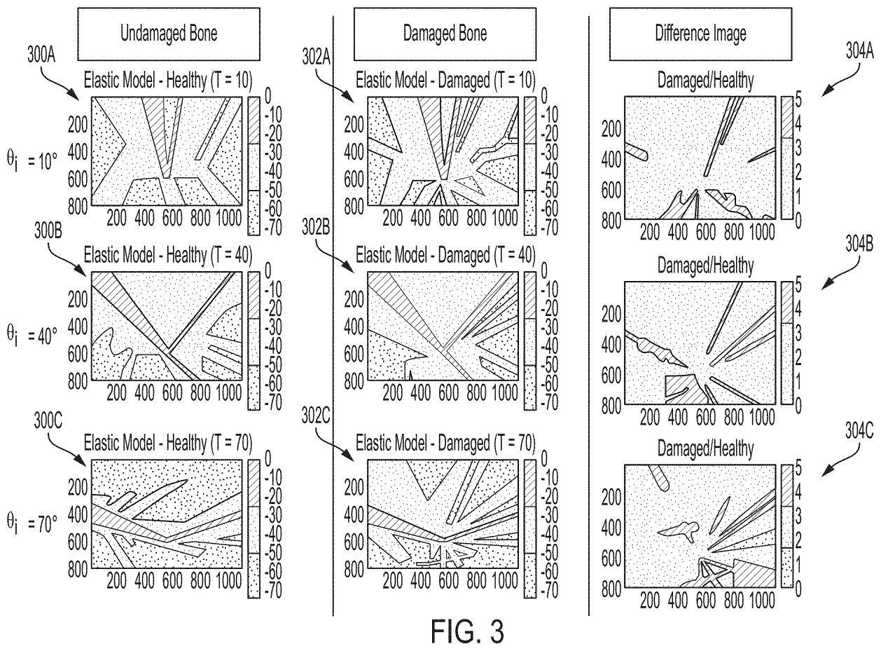

FIG. 3 illustrates example elastic wave scatter simulations from a bone surface, in accordance with some embodiments of the technology described herein. In particular, FIG. 3 illustrates both specular scatter and diffuse scatter in damaged bone and undamaged bone at different angles of incidence for a transmit waves. Plots 300A, 300B, and 300C illustrate scatter simulations in undamaged bone at incident angles of 10°, 40°, and 70° respectively. Acoustic strength of the waves shown in the simulation is shown by crosshatching and the accompanying legends. In some embodiments, the incident angle may be selected to maximize transmission of incident ultrasound waves through a medium (e.g., bone, for example, at an interface between water and bone). Plots 302A, 302B, and 302C illustrate scatter simulations at incident angles of 10°, 40°, and 70°, respectively in damaged bone. Plots 304A, 304B, and 304C illustrate difference images comparing the scatter simulations in undamaged bone and damaged bone for each incident angle. In particular, each of plots 304A, 304B, and 304C illustrate the scatter simulations of damaged bone having the scatter simulations of undamaged bone at the same incident angle subtracted from it.

On bone surface which is damaged and thus exhibits defects (e.g., pitting, cracking, etc.), diffuse scatter is increased and specular reflection is decreased, as shown in plots 304A-304C. The inventors have recognized that measuring both the specular scatter and diffuse scatter from the bone surface and comparing the two types of scatter, for example, using a difference image, can reveal the presence of a stress fracture in bone and/or other hard tissue(s). In some embodiments, a scatter matrix may be used to illustrate scatter from each point on a bone or other object of interest as imaged from each incident (transmit) and exiting (receive) angle.

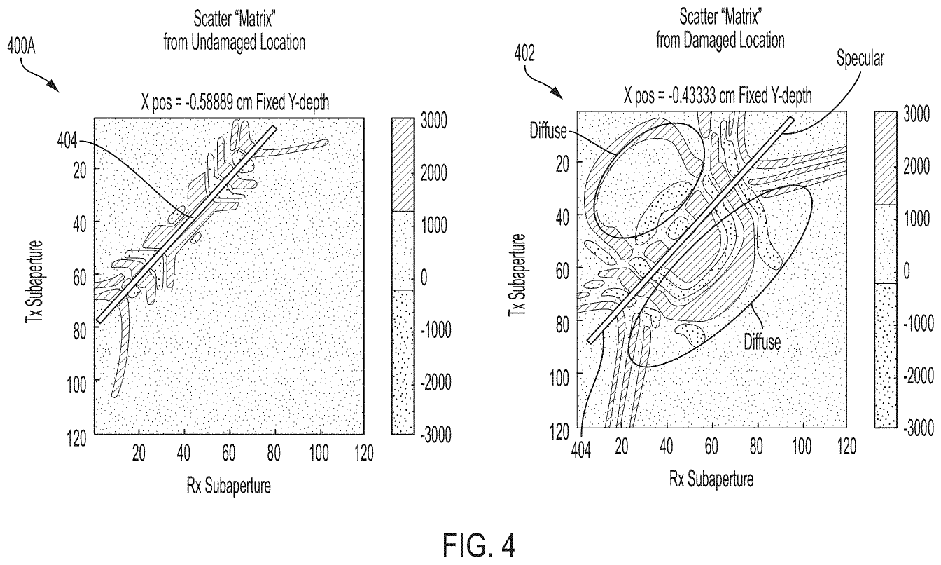

FIG. 4 illustrates example graphs depicting the “scatter matrix” from an ABS tube, in accordance with some embodiments of the technology described herein. Matrix 400 illustrates an example scatter matrix from an undamaged location of the ABS tube. Specular reflection is illustrated by signal along the line 404 while diffuse reflection is shown by signal at other locations on the matrix. As shown in FIG. 4, matrix 400 has a relatively high amount of specular reflection and relatively low amount of diffuse reflection. On the other hand, matrix 402 which illustrates an example scatter matrix from a damaged location of the ABS tube has a relatively low amount of specular reflection and a relatively high amount of diffuse reflection. Thus, a comparison of specular and diffuse scatter as shown by a scatter matrix can identify the existence of a stress fracture in bone and/or other hard tissue(s). In some embodiments, relative amounts of specular and diffuse scatter from a location of interest may be compared to each other to determine the existence of a stress fracture in bone and/or other hard tissue(s).

In some embodiments, relative amounts of specular and diffuse scatter from a location of interest may be compared to amounts of specular and diffuse scatter in a prior acquired image. For example, relative amounts of specular and diffuse scatter from a location of interest may be compared to amounts of specular and diffuse scatter in a prior acquired image of undamaged bone and/or hard tissue(s). A decrease in specular scatter and an increase in diffuse scatter can indicate defects at the bone surface such as pitting or cracking which may indicate the existence of a stress fracture.

In some embodiments, techniques measuring specular and diffuse scatter at a location of interest may further be used to track recovery of a stress fracture. In particular, relative amounts of specular and diffuse scatter from a location of interest may be compared to amounts of specular and diffuse scatter in a prior acquired image at the location of interest to determine whether defects (e.g., pits, scratches) at the bone surface have subsided. For example, an increase in the amount of specular scatter and a decrease in the amount of diffuse scatter may indicate that the bone surface has recovered and it is safe to resume normal activities.

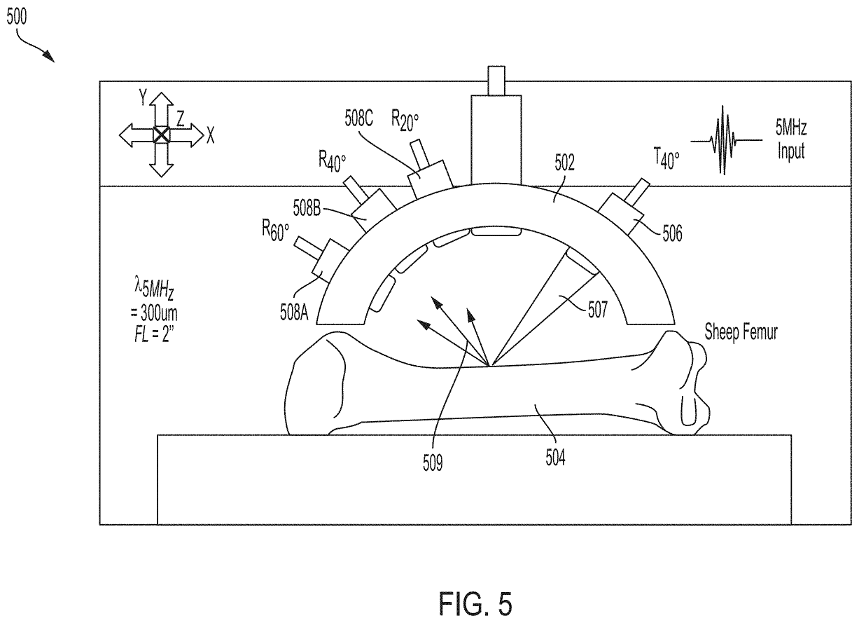

The techniques described herein for enhancing sensitivity in ultrasound imaging may be implemented by any suitable ultrasound imaging apparatus, for example, the apparatus 200 shown in FIG. 2. As described herein, in some embodiments, an apparatus may be configured having transmit and receive transducers configured on one or more different substrates, for example, as shown in FIG. 5. FIG. 5 illustrates an example embodiment of an automated stress fracture diagnosis, ranking, and tracking apparatus, in accordance with some embodiments of the technology described herein. Apparatus 500 shown in FIG. 5 comprises a frame 502 configured to automatically position the apparatus 500 relative to a location of interest. Frame 502 may be configured having the same functions and abilities as the positioner system 202 shown in FIG. 2, in some embodiments. As shown in FIG. 5, the apparatus 500 may be configured for imaging a bone 504. In the illustrated embodiment, the apparatus 500 is configured for imaging a sheep femur. The bone 504 may be disposed in a liquid tank, such as a water tank, to provide a coupling medium which provides an arbitrary scanning range for the apparatus 500 in accordance with some embodiments of the technology described herein.

The apparatus 500 shown in FIG. 5 comprises multiple transmit and receive channels 502, 508A, 508B, 508C. As described herein, the inventors have recognized that it is advantageous to measure both the specular reflection and diffuse reflection from a location of interest to better diagnose, rank, and track suspected stress fractures in bone and/or other hard tissue(s) using ultrasound imaging. While specular reflection occurs when incident waves are reflected at the same angle to the surface normal as the incident ray, diffuse reflection occurs when incident waves are scattered at many different angles to the surface normal. Multiple receive transducers 508A, 508B, 508C configured at different angles (60°, 40°, and 20°, respectively in the illustrated embodiment) may allow for collection of diffuse reflection occurring at a wide range of angles to the surface normal.

Transmit channel 506 may comprise a plurality of transducers configured to perform a transmit function to propagate a beam of transmit waves 507 towards a location of interest. The transmit waves 507 are reflected as shown by the arrows 509 at different angles collected by the receive channels 508A-508C. Receive channels 508A-508C may comprise a plurality of transducers configured to perform a receive function to collect reflected signals from the bone 504. Receive channel 508B is configured to collect reflection from the bone occurring at approximately the same angle incident to the surface normal as the transmit wave (i.e., specular reflection), while receive channels 508A and 508C are configured at other angles such that receive channels 508A and 508C collect diffuse reflection. The inventors have recognized that configuring the transmit and receive transducers on different substrates, and further configuring the receive transducers on a plurality of substrates disposed at different angles may allow for improved collection of both specular and diffuse scatter from the location of interest. However, in some embodiments, the transmit and receive transducers may be disposed on the same substrate, may be disposed in the same housing, and/or may comprise the same transducer configured to perform both transmit and receive functions, and aspects of the technology described herein are not limited in this respect.

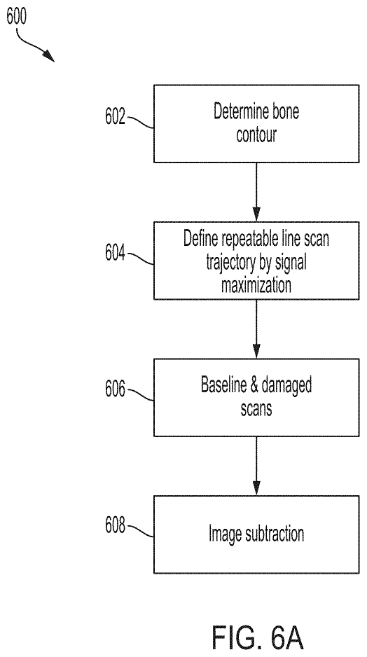

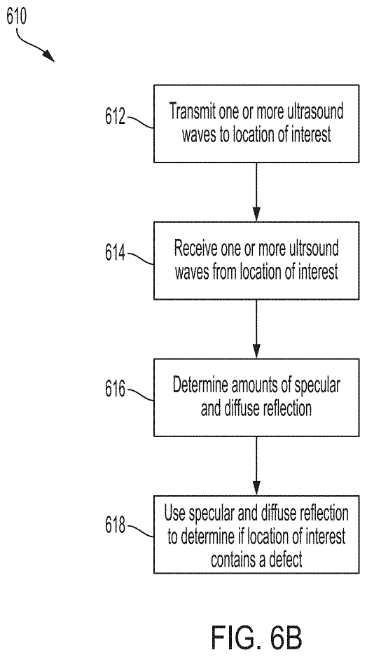

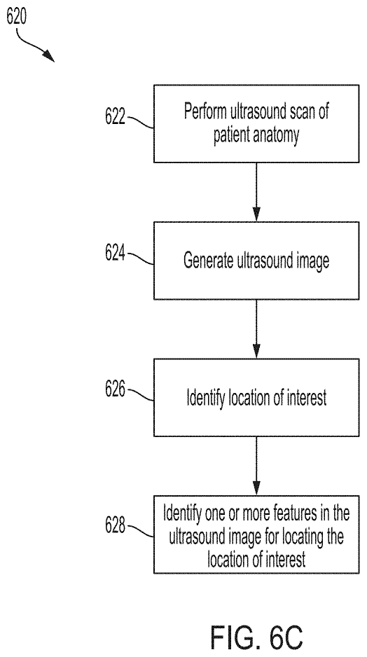

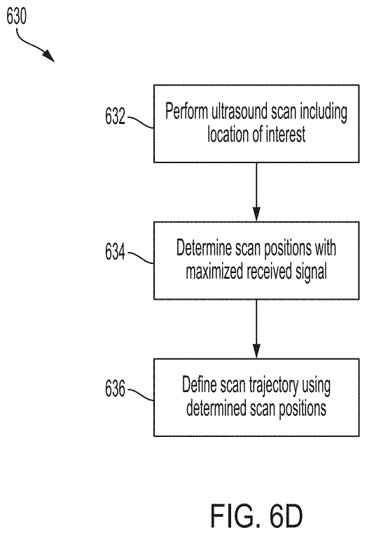

The techniques described herein may be combined in any suitable configuration to perform improved diagnosing, ranking, and tracking of stress fractures in bone and/or other hard tissue(s). In some embodiments, the techniques described herein may be performed as a method. For example, FIGS. 6A-6D illustrate example methods for diagnosing, ranking, and tracking stress fractures in bones and/or other hard tissue(s), in accordance with some embodiments of the technology described herein.

FIG. 6A illustrates an example method 600 for diagnosing, ranking, and tracking stress fractures in bones and/or other hard tissue(s), in accordance with some embodiments of the technology described herein. The method 600 begins at act 602 where a bone contour is determined. For example, the techniques described herein for identifying a location on a bone, in particular, by performing a sweeping scan of the patient anatomy, may be used to determine a bone contour. At act 604, the techniques described herein for automated alignment of the ultrasound examination device and ultrasound transducer elements relative to a location of interest may be performed, in particular, by making adjustments in ultrasound examination device position while monitoring the received ultrasound signal to define a scan trajectory in which ultrasound signal is maximized that can be repeated on the current and/or subsequent scans. At act 606, the scan trajectory may be used to perform a scan of the bone and/or other hard tissue(s) at the location of interest having the suspected stress fracture. The scan (referred to herein as the “damaged scan”) may be compared to previously acquired image data, for example, a baseline image of healthy bone and/or an image acquired earlier in the patient's recovery process. At act 608, the baseline image may be subtracted from an image produced from the damaged scan to produce a difference image. The difference image may illustrate the relative changes in amounts of specular and diffuse reflection, such that a stress fracture at the location of interest can be diagnosed, ranked, and/or tracked, as described herein.

FIG. 6B further illustrates an example method 610 for diagnosing, ranking, and tracking stress fractures in bones and/or other hard tissue(s), in accordance with some embodiments of the technology described herein. The method 610 may facilitate examination of bone and/or other hard tissue(s), including, for example, detection of one or more defects in the bone and/or other hard tissue(s). The method 610 begins at act 612, where one or more ultrasound waves may be transmitted to a location of interest, for example, using one or more ultrasound transducer elements of an ultrasound examination device. At act 614, one or more ultrasound waves may be received from the location of interest. The one or more received ultrasound waves may comprise reflections of at least some of the one or more transmitted ultrasound waves. In some embodiments, the one or more ultrasound waves may be transmitted and received according to the synthetic aperture techniques described herein.

At act 616, the one or more received waves may be used to determine an amount of specular and diffuse reflection received from the location of interest. At act 618, the amounts of specular and diffuse reflection may be used to determine if the location of interest contains a defect (e.g., pitting, cracking, and/or other defects which may be indicative of a stress response and/or stress fracture). Using the amounts of specular and diffuse reflection to determine if the location of interest contains a defect may be performed in any suitable manner. In some embodiments, the amount of specular reflection may be compared to the amount of diffuse reflection to determine if the location of interest contains a defect. In some embodiments, the amount of specular reflection may be compared to a known amount of specular reflection and/or the amount of diffuse reflection may be compared with a known amount of diffuse reflection to determine if the location of interest contains a defect. In some embodiments, it may be determined that that location of interest contains a defect when the amount of specular reflection is less than the known amount of specular reflection and/or when the amount of diffuse reflection is greater than the amount of known diffuse reflection.

FIG. 6C further illustrates an example method 620 for diagnosing, ranking, and tracking stress fractures in bones and/or other hard tissue(s), in accordance with some embodiments of the technology described herein. For example, FIG. 6C illustrates a method 620 which may be used to identify a location of interest on a bone and/or other hard tissue. Method 620 begins at act 622, where an ultrasound scan of a patient anatomy may be performed, for example, using the ultrasound examination techniques described herein. In some embodiments, the patient anatomy may contain the location of interest. At act 624, an ultrasound image may be generated using the obtained ultrasound scan. At act 626, a location of interest may be identified, for example, using the generated ultrasound image and/or ultrasound scan. At act 628, one or more features in the ultrasound image may be identified. In some embodiments, the one or more features may be used to localize the location of interest. In some embodiments, the one of more features may be used to locate the location of interest and thereafter a subsequent ultrasound scan of the location of interest may be performed. In some embodiments, the one or more features may be stored in memory, for example in a database of an ultrasound examination system

FIG. 6D further illustrates an example method 630 for diagnosing, ranking, and tracking stress fractures in bones and/or other hard tissue(s), in accordance with some embodiments of the technology described herein. In some embodiments, the method 630 may be used to position an ultrasound examination device relative to a location of interest in a patient. The method 630 begins at act 632 where an ultrasound scan may be performed. The ultrasound scan may be performed at a region including at least the location of interest. At act 634, one or more scan positions in which a received signal of the ultrasound scan is maximized may be determined. At act 636, a scan trajectory using the determined scan positions may be defined. For example, the scan trajectory may comprise at least one or more of the scan positions in which the received signal of the ultrasound scan is maximized. In some embodiments, the scan trajectory may be stored in memory, for example, in a database of an ultrasound examination system. In some embodiments, the scan trajectory may be used to perform a subsequent ultrasound scan. In some embodiments, an automated positioning system may be used to move an ultrasound examination device along the scan trajectory. The ultrasound examination device may further transmit and/or receive one or more ultrasound waves to/from the location of interest at one or more of the plurality of scan positions along the scan trajectory.

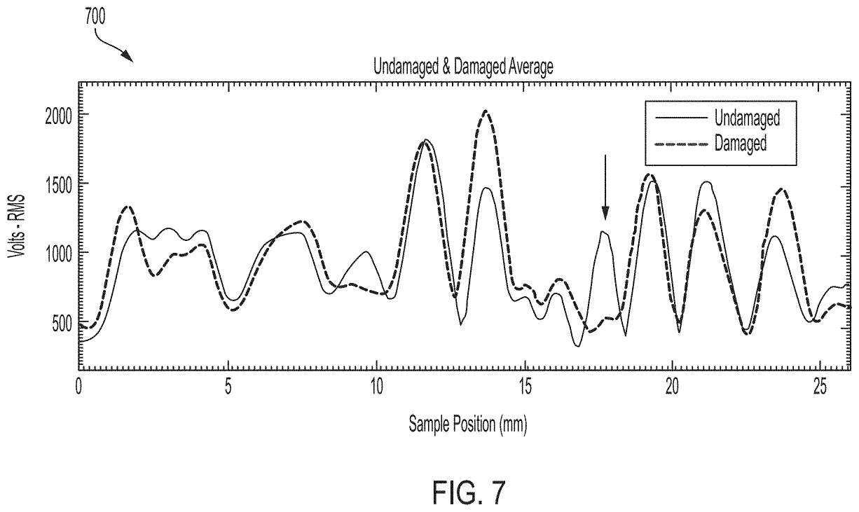

FIG. 7 illustrates an example graph 700 illustrating the difference in receive ultrasound signal between damaged bone and undamaged bone. In particular, graph 700 illustrates an example of a change in received signal between a baseline scan of healthy bone and a scan of damaged bone, as described in act 606 of example method 600. As shown in graph 700, a location at which the undamaged scan signal and damaged scan signal differ may be identified. Such location may be indicative of a location where relative amounts of specular and/or diffuse scatter differ between undamaged and damaged bone and thus a location indicating the presence of a stress fracture.

In some embodiments, the frequency content of scatter from damaged bone and/or undamaged bone may be analyzed to detect sub-wavelength scale damage in bone and/or other hard tissue by measuring both the frequency-dependent diffuse and specular scatter and making a comparison between the two. The inventors have recognized that excitation frequency may be a factor in detecting small defects at the bone surface. Higher frequencies may be more sensitive to smaller defects, but may provide less penetration into bone. Lower frequencies may be less sensitive to smaller defects, but may greater signal-to-noise ratio into bone. Thus, collecting signal across a plurality of frequencies may be advantageous to ensure sub-wavelength scale features at the bone surface are detected by an ultrasound scan.

Such a frequency-dependent technique may be achieved by transmitting a broadband pulse having a continuous range of frequencies to the location of interest, collecting the reflected signal through receive channels, and calculating a Fourier transform of the received signal. Acquiring such a data set is very rapid, scaling as the number of elements times the round-trip sonic transit time (in some embodiments, comprising 10 ms total). The same data set can be used first to locate the bone surface, and then analyze the frequency-dependent back and oblique scatter from a point of interest.

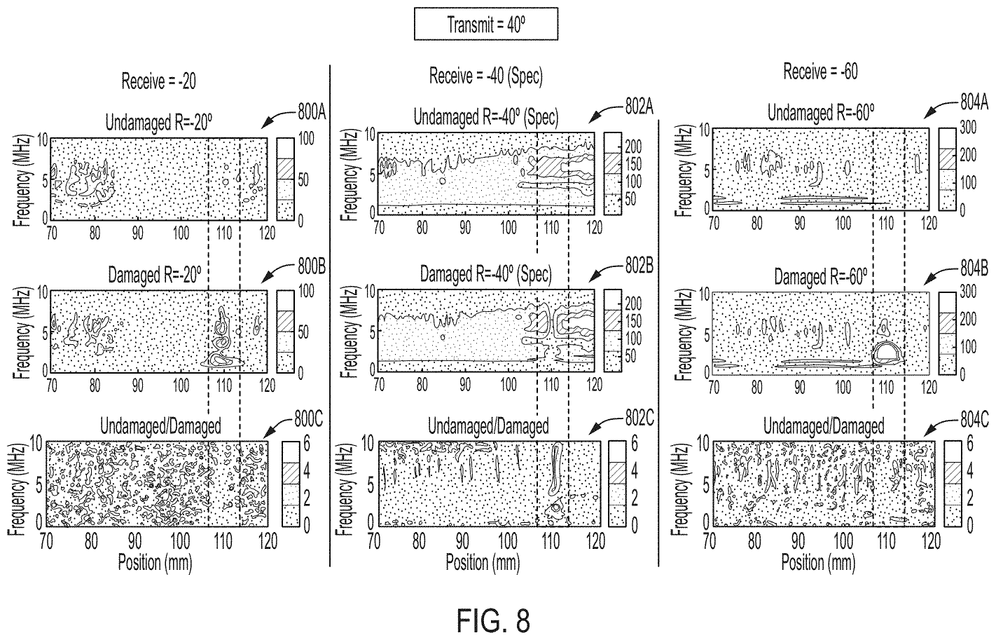

FIG. 8 illustrates a series of images depicting the frequency content of scatter from a lesion on the bone surface, in accordance with some embodiments of the technology described herein. Graphs 800A, 802A, and 804A illustrate frequency scatter plots of undamaged bone at different receive angles (−20°, −40°, and −60° respectively). Graphs 800B, 802B, and 804B illustrate frequency scatter plots of damaged bone at different receive angles (−20°, −40°, and −60° respectively). The transmit angle for each of the plots is 40°. Thus, plots 802A and 802B illustrate collected specular scatter and 800A, 800B, 804A, and 804B illustrate collected diffuse scatter. Plots 800C, 802C, and 804C illustrate difference images of the undamaged and damaged plots for each receive angle. The dotted lines for each plot illustrate the location of a bone lesion causing one or more defects at the bone surface. The plots shown in FIG. 8 illustrate that the presence of the bone lesion can be detected and localized by examining the relative amounts of specular and diffuse scatter between damaged and undamaged bones. For example, the diffuse scatter is shown to increase in damaged bone, as shown by plots 800C and 804C while the specular reflection is shown to decrease in damaged bone, as shown by plot 802C. The x-axis tracks position in millimeters, allowing for the defect in in the bone surface to be localized, for example, as shown in FIG. 8, to 110 mm in the scan trajectory.

The inventors have demonstrated the viability of the techniques described herein for diagnosing, ranking, and tracking of stress fractures in bone and/or other hard tissue(s) through multiple experiments, the results of which are shown herein.

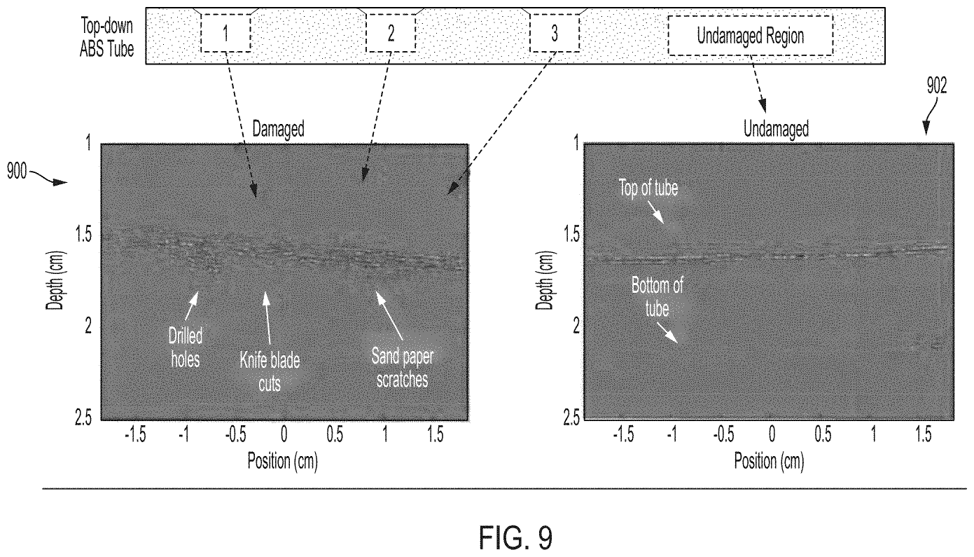

FIG. 9 illustrates example ultrasound images showing specular and diffuse reflection in damaged and undamaged ABS pipe, in accordance with some embodiments of the technology described herein. FIG. 9 illustrates the results of an experiment conducted to image, using ultrasound imaging, portions of ABS pipe. As shown in FIG. 9, portions of the ABS pipe were damaged using different methods (e.g., with drill holes (1), knife blade cuts (2), and sandpaper scratches (3)). In addition, a portion of the ABS pipe was left undamaged. Image 900 illustrates an acquired ultrasound image of the damaged portion of the pipe containing the drill holes (1), knife blade cuts (2), and sandpaper scratches (3), while image 902 illustrates an image of the undamaged portion of the ABS pipe. As can be seen by comparing the two images 900, 902, even with relatively low image contrast, the damage sites (1), (2), and (3) can be detected in image 900. Image 900 illustrates an interruption in the specular reflection at the damage sites, while the undamaged portion is able to be imaged without interruptions. Thus, the damage sites can be detected and localized.

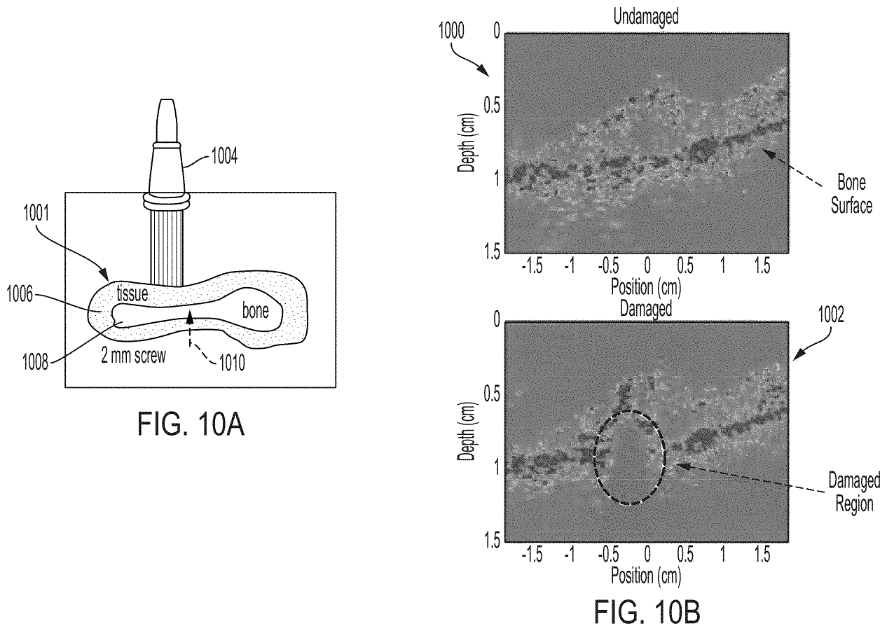

FIG. 10A illustrates an example experimental setup for measuring specular and diffuse reflection in a damaged sheep bone, in accordance with some embodiments of the technology described herein. The example shown in FIG. 10A illustrates an object to be imaged, which, in the illustrated embodiment, is a sheep bone 1008 having tissue 1006 thereon. A screw 1010 has been inserted into the underside of the bone 1008 through tissue 1006. An ultrasound examination device 1004 is used to image the bone 1008. Although in the illustrated embodiment the ultrasound examination device is shown having transmit and receive channels housed in a single housing, in some embodiments, the transmit and receive transducers may be disposed on separate substrates and/or in different housings (e.g., being disposed at different angles, as shown in FIG. 5).

FIG. 10B illustrates example ultrasound images showing specular and diffuse reflection in damaged and undamaged sheep bone, in accordance with some embodiments of the technology described herein. FIG. 10B illustrates images 1000, 1002 of the object 1001 acquired before and after the damage was introduced by inserting the screw 1010 into the underside of the bone 1010. Image 1000 illustrates specular reflection received from the bone surface being fairly constant along the bone surface. By contrast, image 1002 illustrates a disruption in specular reflection in the middle of the bone surface, as shown by the outlined circle labeled “damaged region.” The disruption in specular reflection indicates the presence of a potential defect in the bone 1010.

The results of the experiment shown in FIGS. 10A-10B illustrates that the techniques described herein for diagnosing, ranking and tracking stress fractures in bone and/or other hard tissue(s) is possible even where there is tissue surrounding the bone and/or other hard tissue(s). As described herein, although ultrasound imaging was previously used for imaging soft tissue, the techniques described herein allow for imaging of bone and/or other hard tissue(s) to diagnose, rank, and track stress fractures. In particular, the synthetic aperture approach allows for flexible data processing, including selecting a focal point of the waves after-the-fact of imaging. In addition, transmitting a broadband pulse comprising a plurality of frequencies across a continuous spectrum allows for study of a range of frequencies, including lower frequencies which may provide deeper penetration into tissue and/or bone. Furthermore, the results shown in FIG. 10B illustrate that defects existing in and/or on the underside of bone are also detectable using the techniques described herein, and detection is not limited to defects on the surface of the bone.

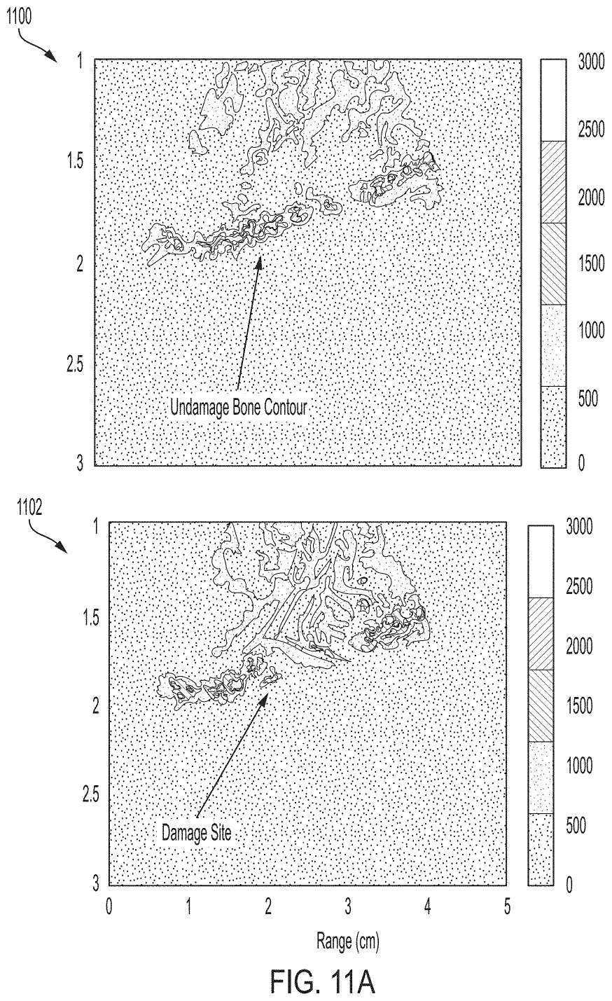

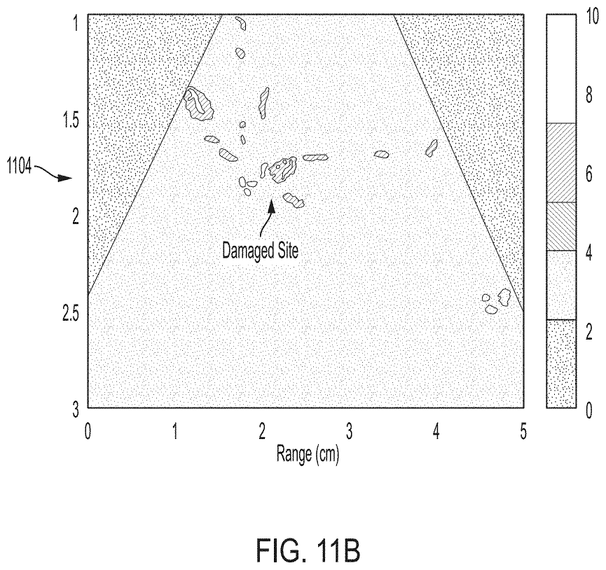

FIG. 11A illustrates example ultrasound images showing specular and diffuse reflection in damaged and undamaged sheep bone, in accordance with some embodiments of the technology described herein. FIGS. 11A-11B include a similar experimental setup as described with respect to FIGS. 10A-10B. In particular, plot 1100 is a scatter matrix of signal collected from an undamaged bone, while plot 1102 is a scatter matrix of the bone after damage has been introduced (e.g., by inserting a screw into the underside of the bone). As can be seen by comparing plots 1100 and 1102, the specular reflection has decreased in the damaged bone at the damage site. FIG. 11B illustrates an example difference image between ultrasound images of damaged and undamaged sheep bone, in accordance with some embodiments of the technology described herein. The difference image 1104 more clearly shows the damage site as having a relative difference in the amounts of specular reflection measured between undamaged bone and damaged bone.

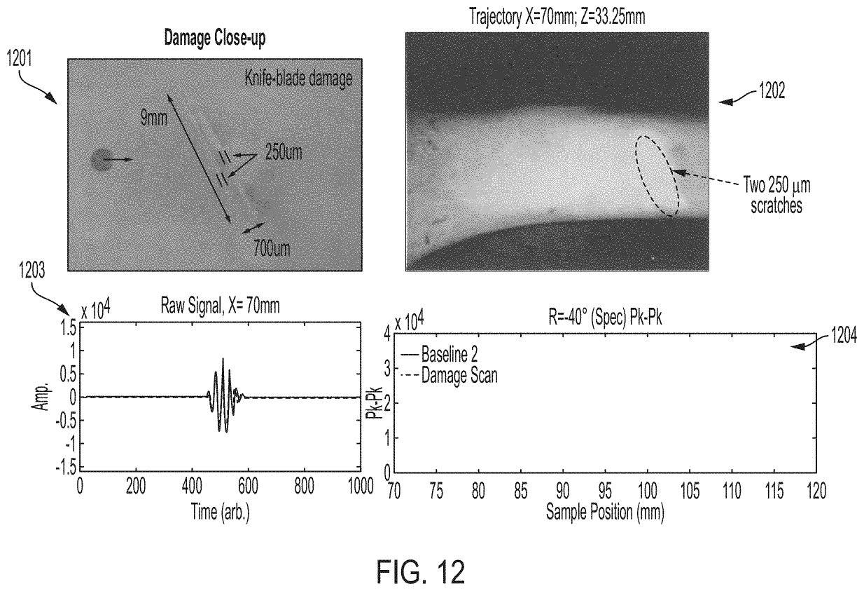

Further illustration of the viability of the techniques described herein for diagnosing, ranking, and tracking of stress fractures in bone and/or other hard tissue(s) through multiple experiments are shown in FIGS. 12-23. FIGS. 12-23 are a series of graphs and illustrations depicting the use of the devices and methods provided herein in various studies to perform measurements with phantoms, e.g., plastic bone simulants, excised bare animal bones (i.e., with the tissue(s) removed), and excised intact animal limbs (i.e., with the tissue(s) intact), in accordance with some embodiments of the technology described herein. These measurements have demonstrated the ability of the devices and methods provided herein to detect small changes in the bone surface, even through tissue.

These working examples demonstrate further that sub-wavelength (e.g., <300 μm) damage features on the surface of a smooth ABS (plastic) tube and/or on a bone can be easily detected using the techniques described herein, for example, by measuring both the frequency-dependent diffuse non-specular and the frequency-dependent specular acoustic scatter from a location of interest. Specular and diffuse scatter can be measured using a linear-array transducer using a synthetic aperture approach. Overall bone contour can be delineated by doing an automated scan of a linear array transducer along a bone.

FIG. 12 is an example of an experimental set up in which damage is introduced to a sample and thereafter imaged. For example, as shown in images 1201 and 1202, damage is introduced to the surface of a bone using a knife blade. The knife blade produces scratches having a width of approximately 250 μm and a length of approximately 9 mm, but these lengths are non-limiting as other lengths are envisioned. The scratches are separated by a distance of 700 μm as a nonlimiting example. Plot 1203 illustrate a plot of received ultrasound signal over time.

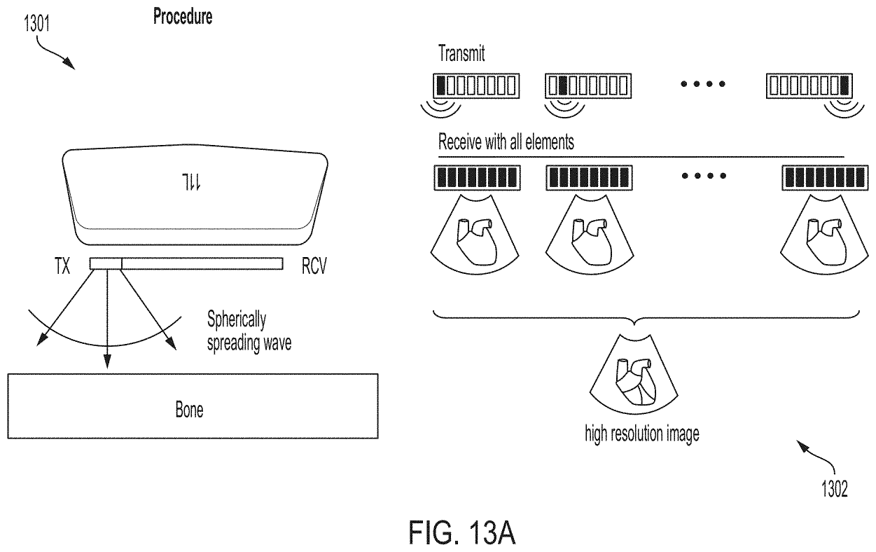

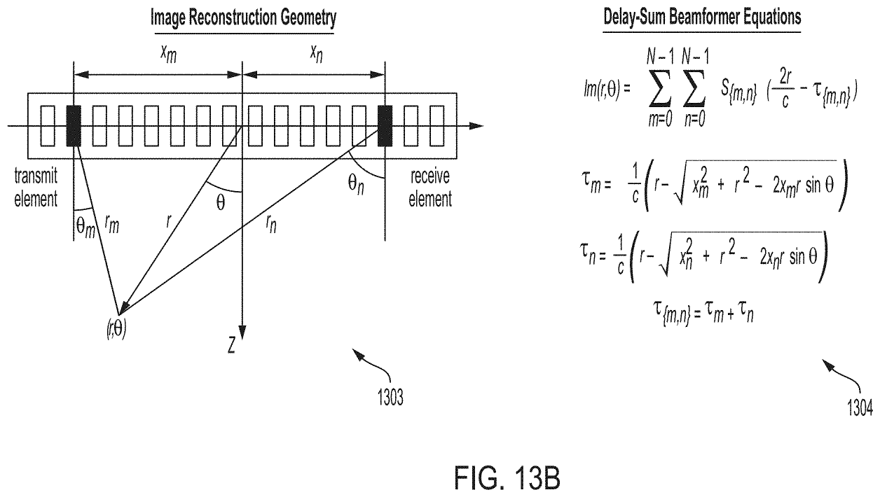

FIGS. 13A-13B illustrates an example configuration for the synthetic aperture techniques described herein. For example, block 1301 illustrates a transmit pattern for an array of transducers. Block 1301 illustrates the operation of the synthetic aperture approach, in particular, illustrating activation of individual transmit elements at discrete times while continuously receiving reflected signal from all receive elements. Block 1303 illustrates the image reconstruction geometry using the synthetic aperture approach and block 1304 gives a delay-sum beamformer equation for image reconstruction based on the geometry given in block 1303.

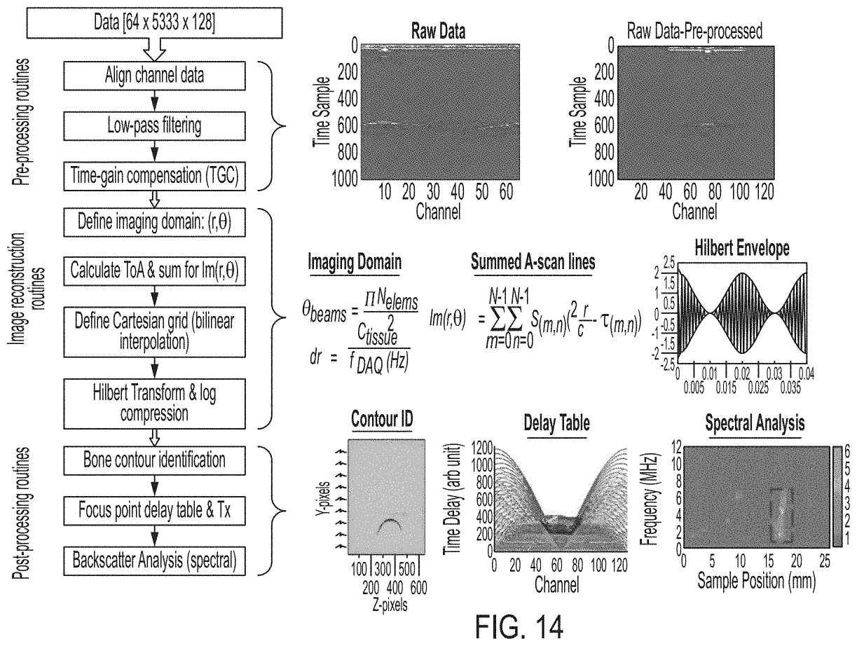

FIG. 14 illustrates example techniques for processing data received using the synthetic aperture techniques described herein.

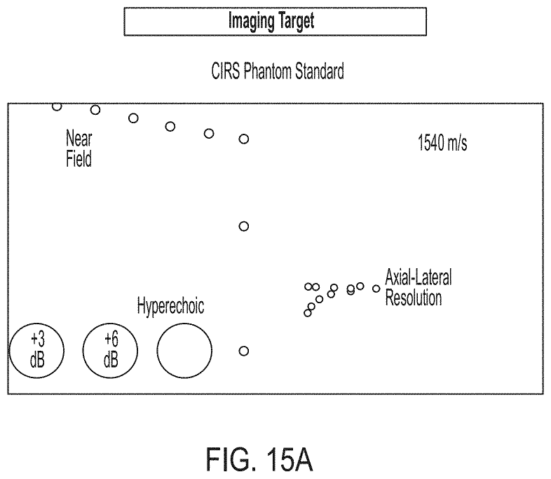

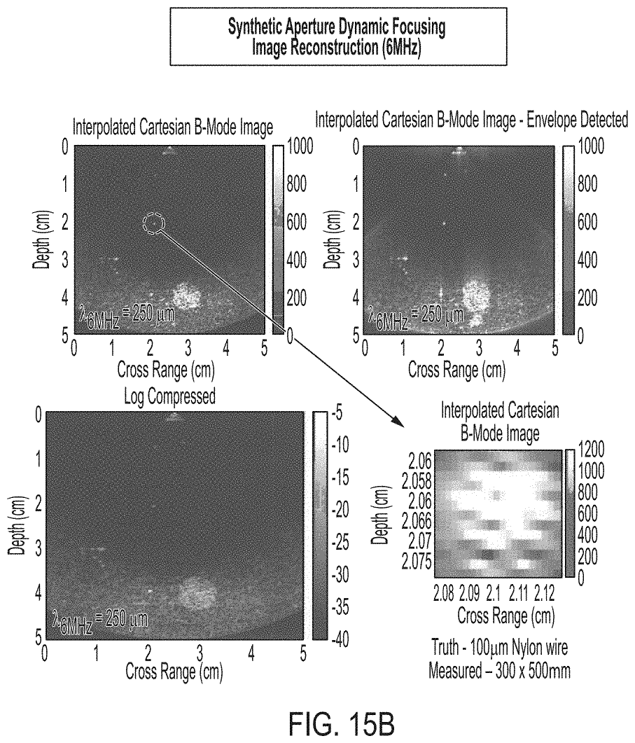

FIGS. 15A-15B illustrates example results acquired using the synthetic aperture techniques described herein. FIG. 15A illustrates a schematic illustration of the objects imaged. FIG. 15B illustrates example ultrasound images of various objects, including, for example, a 100 μm nylon wire, using the techniques described herein.

FIGS. 16A-16C illustrates example results of received ultrasound signal at various focus depths. For example, FIG. 16A illustrates various focus depths along a lamb shank bone (1-4). FIGS. 16B-16C illustrates the collected ultrasound signal as a function of time at various focus depths and a frequency domain plot of the same. Thus, FIGS. 16A-16C illustrates that image acquisition at the various focus depths may be achieved by the techniques described herein. In particular, it is necessary to reduce and/or eliminate tissue interference when imaging bone and/or other hard tissue(s). In some embodiments, controlling a focus depth at which imaging is performed facilitates elimination of tissue interference such that the bone and/or other hard tissue(s) may be imaged.

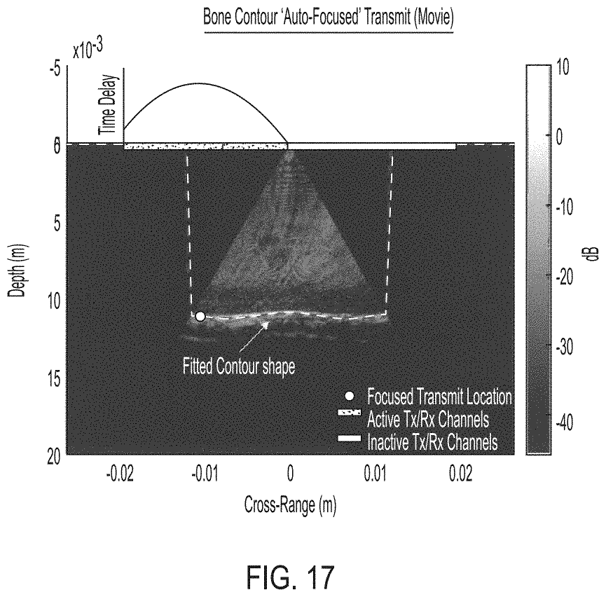

FIG. 17 illustrates an example ultrasound image used to determine focus depths to perform imaging. For example, FIG. 17 illustrates that focus depths may be determined based on fitting a contour to the objects imaged (e.g., a bone). Further, in some embodiments, the techniques described herein for identifying a location on a bone include mapping a bone contour at or around a location of interest. FIG. 17 illustrates that a mapping of bone contour may be achieved using the techniques described herein.

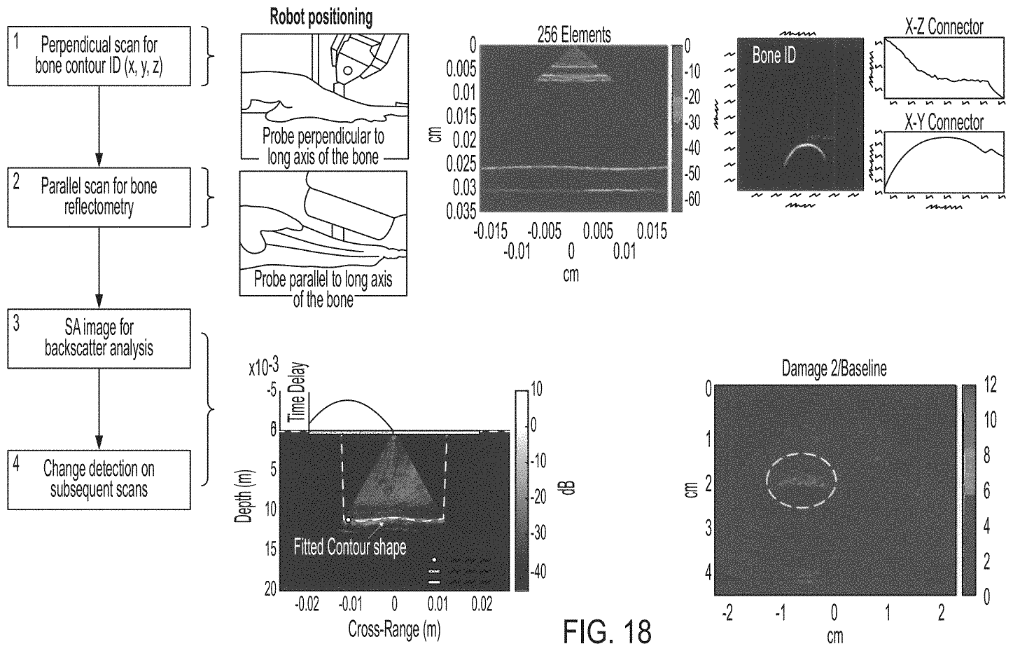

FIG. 18 illustrates example process of implementing the techniques described herein. For example, FIG. 18 illustrates a process of producing a mapping of bone contour, locating a location of interest, and performing a backscatter analysis based on acquired synthetic aperture data. FIG. 18 further illustrates the results of such a process.

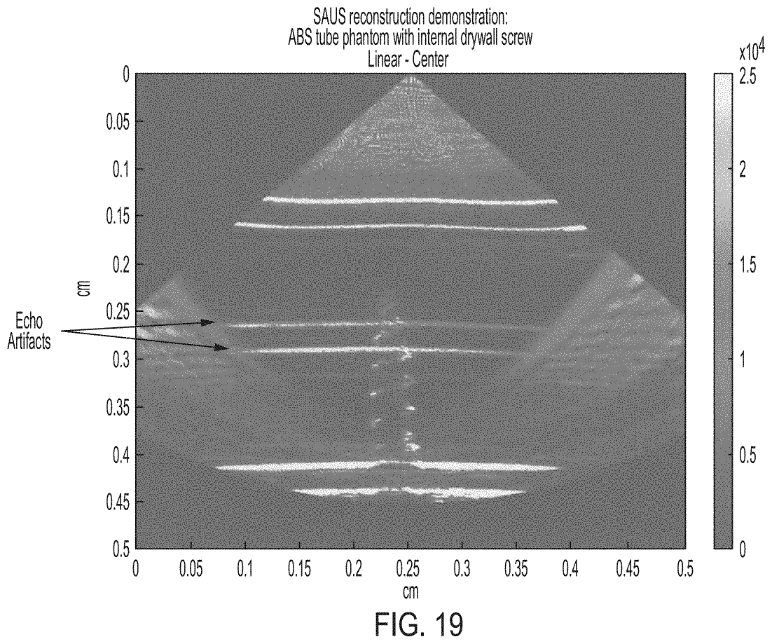

FIG. 19 illustrates an example ultrasound image of a PVC tube using the synthetic aperture techniques described herein, the PVC tube having damage introduced therein. For example, prior to imaging, a screw was inserted into an underside of the PVC tube. The acquired image illustrates detection of the screw having a high level of detail. FIG. 19 illustrates the ability to detect sub-wavelength scale features using the techniques described herein. Such enhanced sensitivity may be necessary to detect small features (e.g., cracking, pitting, etc.) at the bone surface indicative of a stress fracture.

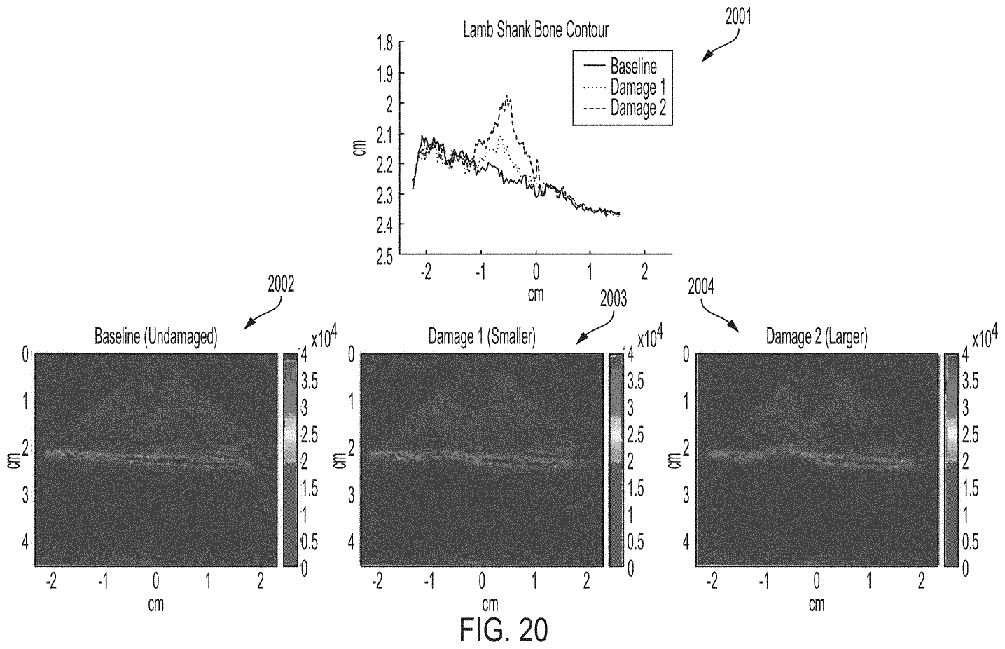

FIG. 20 illustrates example ultrasound images acquired from a sheep femur having intact tissue using the synthetic aperture techniques described herein. The images shown in FIG. 20 illustrate three cases—where the bone is undamaged, where the bone exhibits a small amount of damage, and where the bone exhibits a larger amount of damage. In the illustrated embodiment, damage was introduced to the femur using a screw inserted into an underside of the bone. A contour of the bone was acquired, as shown in block 2001, for each case. Ultrasound images for each case are shown in blocks 2002-2004. Blocks 2001-2004 illustrate a detectable change in the contour of the bone due to the introduced damage. FIG. 20 illustrates the ability to distinguish between varying levels of damage in an object, using the techniques described herein. The ability to do so may provide for improved ranking of stress fractures in bone and/or other hard tissue(s). The methods applied in the embodiment shown in FIG. 20 may be applied to diagnosing, ranking, and/or tracking of a stress fracture, for example, by examining changes to bone contour due to the formation of a callus, as described herein.

Various example implementations of the techniques described herein are further shown, for example, in U.S. provisional patent application Ser. No. 62/858,742, entitled “ULTRASONIC METHODS FOR TRACKING STRESS FRACTURES OF BONE”, filed Jun. 7, 2019 under Attorney Docket No. M0437.70149US00, which is hereby incorporated by reference in its entirety.

It should be understood that these studies shown in the figures, while indicating exemplary embodiments of the invention, are given by way of illustration only. From the above discussion and these studies, one skilled in the art can ascertain the essential characteristics of this invention, and without departing from the spirit and scope thereof, can make various changes and modifications of the invention to adapt it to various uses and conditions.

(5) Methods Using Signature Characteristics of Stress Fractures

As described herein, stress fractures exhibit certain characteristics during the recovery process which may be used to better diagnose, rank, and track stress fractures using ultrasound techniques. For example, stress fractures begin with a stress reaction in which edema within bone marrow causes increased pressure and increased diffusion of fluid through the bone structure, creating what is known as a “bone bruise” which may be exhibited as pitting on the surface of the bone. The inflammation caused by the stress response causes rapid remodeling within the bone at the bone surface, and the bone is weakened due to remodeling. Larger fractures, which may be detectable through imaging, occurs at points where the bone is weakened. During recovery, a callus forms on the bone surface and subsides after full recovery.

The inventors have recognized that such signature characteristics exhibited during stress fracture incidence and recovery may be used to better diagnose, rank, and track stress fractures in bone and/or other hard tissue(s). According to some embodiments of the technology described herein, signature characteristics can be used by using the ultrasound imaging techniques described herein to: (1) detect edema within bone marrow; (2) detect pitting and/or other defects at the bone surface by, for example, comparing relative amounts of specular and diffuse scatter from a location of interest; (3) detect weakened bone by examining reduced sound speed and reflectivity from the location of interest; (4) examine larger fractures visible in an ultrasound image, for example, by using the SA approach described herein; (5) examine and track the development of a callus at the bone surface using acquired ultrasound images; (6) detect increased blood flow at or near the location of interest, for example, by using clinical doppler ultrasound.

For example, examining and tracking the development of a callus at the bone surface using acquired ultrasound images may facilitate improved diagnosis, ranking, and/or tracking of stress fractures in bone and/or other hard tissue(s). Once a stress fracture occurs, healing will involve the formation of a “callus” around the fracture, which can be monitored using ultrasound. Once the fracture is completely healed, the callus will subside. Accurate monitoring of the progression of the callus (e.g., the size) is another signature that the devices and methods provided herein use to monitor recovery. In some embodiments, the devices and methods use a linear array transducer to measure the extent of the callus from the overall bone surface, for example, by imaging the callus and analyzing the acquired image (e.g., to determine the size of the callus, for example, as compared to a known size or prior acquired data). Tracking the extent of the callus around a stress fracture may provide for improved accuracy in estimating recovery timelines, including determining when it is safe to resume normal weight bearing activities.

Although aspects of the technology described herein have been described with respect to diagnosing, ranking, and tracking stress fractures in bones, the techniques described herein may be applied in other contexts, for example, other contexts which include imaging of bone surfaces, such as the study of bone lesions, for example.

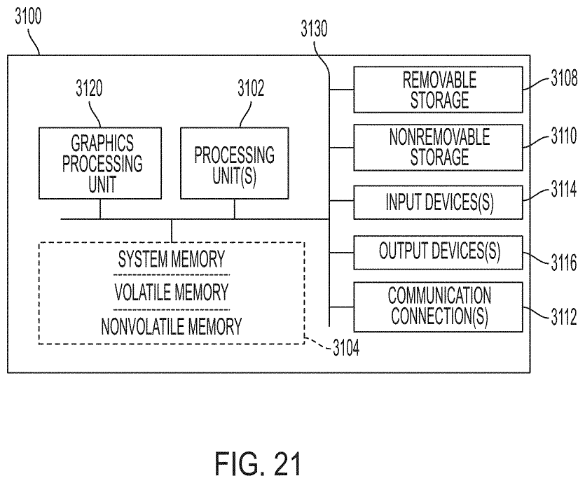

The data analysis described herein can be implemented using a computer system such as described in connection with FIG. 21 and/or using special purpose logic circuitry. FIG. 21 is a block diagram of a general-purpose computer which processes computer program code using a processing system. Computer programs on a general-purpose computer generally include an operating system and applications. The operating system is a computer program running on the computer that manages access to various resources of the computer by the applications and the operating system. The various resources generally include memory, storage, communication interfaces, input devices and output devices.

Examples of such general-purpose computers include, but are not limited to, larger computer systems such as server computers, database computers, desktop computers, laptop and notebook computers, as well as mobile or handheld computing devices, such as a tablet computer, hand held computer, smart phone, media player, personal data assistant, audio and/or video recorder, or wearable computing device.

With reference to FIG. 21, an example computer 3100 comprises a processing system including at least one processing unit 3102 and a memory 3104. The computer can have multiple processing units 3102 and multiple devices implementing the memory 3104. A processing unit 3102 can include one or more processing cores (not shown) that operate independently of each other. Additional co-processing units, such as graphics processing unit 3120, also can be present in the computer. The memory 3104 may include volatile devices (such as dynamic random access memory (DRAM) or other random access memory device), and non-volatile devices (such as a read-only memory, flash memory, and the like) or some combination of the two, and optionally including any memory available in a processing device. Other memory such as dedicated memory or registers also can reside in a processing unit. This configuration of memory is illustrated in FIG. 21 by dashed line 3104. The computer 3100 may include additional storage (removable and/or non-removable) including, but not limited to, magnetically-recorded or optically-recorded disks or tape. Such additional storage is illustrated in FIG. 21 by removable storage 3108 and non-removable storage 3110. The various components in FIG. 21 are generally interconnected by an interconnection mechanism, such as one or more buses 3130.

A computer storage medium, including non-transitory computer storage mediums, is any medium in which data can be stored in and retrieved from addressable physical storage locations by the computer. Computer storage media includes volatile and nonvolatile memory devices, and removable and non-removable storage devices. Memory 3104, removable storage 3108 and non-removable storage 3110 are all examples of computer storage media. Some examples of computer storage media are RAM, ROM, EEPROM, flash memory or other memory technology, CD-ROM, digital versatile disks (DVD) or other optically or magneto-optically recorded storage device, magnetic cassettes, magnetic tape, magnetic disk storage or other magnetic storage devices. Computer storage media and communication media are mutually exclusive categories of media.

The computer 3100 may also include communications connection(s) 3112 that allow the computer to communicate with other devices over a communication medium. Communication media typically transmit computer program code, data structures, program modules or other data over a wired or wireless substance by propagating a modulated data signal such as a carrier wave or other transport mechanism over the substance. The term “modulated data signal” means a signal that has one or more of its characteristics set or changed in such a manner as to encode information in the signal, thereby changing the configuration or state of the receiving device of the signal. By way of example, and not limitation, communication media includes wired media such as a wired network or direct-wired connection, and wireless media include any non-wired communication media that allows propagation of signals, such as acoustic, electromagnetic, electrical, optical, infrared, radio frequency and other signals. Communications connections 3112 are devices, such as a network interface or radio transmitter, that interface with the communication media to transmit data over and receive data from signals propagated through communication media.

The communications connections can include one or more radio transmitters for telephonic communications over cellular telephone networks, and/or a wireless communication interface for wireless connection to a computer network. For example, a cellular connection, a Wi-Fi connection, a Bluetooth connection, and other connections may be present in the computer. Such connections support communication with other devices, such as to support voice or data communications.

The computer 3100 may have various input device(s) 3114 such as a various pointer (whether single pointer or multi-pointer) devices, such as a mouse, tablet and pen, touchpad and other touch-based input devices, stylus, image input devices, such as still and motion cameras, audio input devices, such as a microphone. The computer may have various output device(s) 3116 such as a display, speakers, printers, among other output devices, for example, also may be included.

The various storage 3110, communication connections 3112, output devices 3116 and input devices 3114 can be integrated within a housing of the computer, or can be connected through various input/output interface devices on the computer, in which case the reference numbers 3110, 3112, 3114 and 3116 can indicate either the interface for connection to a device or the device itself as the case may be.

An operating system of the computer typically includes computer programs, commonly called drivers, which manage access to the various storage 3110, communication connections 3112, output devices 3116 and input devices 3114. Such access generally includes managing inputs from and outputs to these devices. In the case of communication connections, the operating system also may include one or more computer programs for implementing communication protocols used to communicate information between computers and devices through the communication connections 3112.

Any of the foregoing aspects may be embodied as a computer system, as any individual component of such a computer system, as a process performed by such a computer system or any individual component of such a computer system, or as an article of manufacture including computer storage in which computer program code is stored and which, when processed by the processing system(s) of one or more computers, configures the processing system(s) of the one or more computers to provide such a computer system or individual component of such a computer system.

Embodiments of the above-described techniques can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Such processors may be implemented as integrated circuits, with one or more processors in an integrated circuit component, including commercially available integrated circuit components known in the art by names such as CPU chips, GPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

The terms “program” or “software” are used herein in a generic sense to refer to any type of computer code or set of computer-executable instructions that can be employed to program a computer or other processor to implement various aspects of the technology described herein as described above. Additionally, it should be appreciated that according to one aspect of this embodiment, one or more computer programs that when executed perform methods of the technology described herein need not reside on a single computer or processor, but may be distributed in a modular fashion amongst a number of different computers or processors to implement various aspects of the technology described herein.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various aspects of the technology described herein may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the technology described herein may be embodied as a method, of which an example has been provided, including FIGS. 6A-6D, and their accompanying descriptions. The acts performed as part of the method may be ordered in any suitable way. Accordingly, embodiments may be constructed in which acts are performed in an order different than illustrated, which may include performing some acts simultaneously or concurrently, even though shown as sequential acts in illustrative embodiments.

Various events/acts are described herein as occurring or being performed at a specified time. One of ordinary skill in the art would understand that such events/acts may occur or be performed at approximately the specified time.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

The terms “substantially”, “approximately”, and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Having thus described several aspects of at least one embodiment of the technology described herein, it is to be appreciated that various alterations, modifications, and improvements will readily occur to those skilled in the art. Such alterations, modifications, and improvements are intended to be part of this disclosure, and are intended to be within the spirit and scope of the present disclosure. Further, though advantages of some embodiments of the technology described herein are indicated, it should be appreciated that not every embodiment will include every described advantage. Some embodiments may not implement any features described as advantageous herein and in some instances. Accordingly, the foregoing description and drawings are by way of example only.

All references, patents, patent applications or other documents cited are hereby incorporated by reference herein.

What is claimed is:

1. A method for examining bone and/or hard tissue of a patient using ultrasound imaging, the method comprising:
transmitting one or more ultrasound waves to a location of interest;
receiving one or more ultrasound waves from the location of interest, the one or more received ultrasound waves comprising reflections of at least some of the one or more transmitted ultrasound waves;
determining an amount of received ultrasound waves comprising specular reflection and an amount of received ultrasound waves comprising diffuse reflection; and
using the determined amounts of specular reflection and diffuse reflection to determine if the location of interest contains a defect.

2. The method of claim 1, further comprising;
comparing the amount of specular reflection to the amount of diffuse reflection; and
using the comparison to determine if the location of interest contains a defect.

3. The method of claim 1, wherein using the determined amounts of specular reflection and diffuse reflection to determine if the location of interest contains a defect comprises:
comparing the amount of specular reflection with a known amount of specular reflection and/or comparing the amount of diffuse reflection with a known amount of diffuse reflection.

4. The method of claim 3, wherein using the determined amounts of specular reflection and diffuse reflection to determine if the location of interest contains a defect comprises:
determining whether the amount of specular reflection is less than the known amount of specular reflection; and
when it is determined that the amount of specular reflection is less than the known amount of specular reflection, making a determination that the location of interest contains a defect.

5. The method of claim 3, wherein using the determined amounts of specular reflection and diffuse reflection to determine if the location of interest contains a defect comprises:
determining whether the amount of diffuse reflection is greater than the known amount of diffuse reflection; and
when it is determined that the amount of diffuse reflection is greater than the known amount of diffuse reflection, making a determination that the location of interest contains a defect.

6. The method of claim 1, wherein transmitting the one or more ultrasound waves comprises transmitting a plurality of ultrasound waves having one or more different frequencies.

7. The method of claim 6, further comprising:
calculating a Fourier transform of the received ultrasound waves for a plurality of frequencies.

8. The method of claim 1, wherein transmitting the one or more ultrasound waves is performed at least in part by a first array of ultrasonic transducers and receiving the one or more ultrasound waves is performed at least in part by a second array of ultrasonic transducers, wherein the first array of ultrasonic transducers is disposed on a first substrate, and the second array of ultrasonic transducers is disposed on a second substrate.

9. The method of claim 1, wherein the defect comprises a stress fracture and/or a stress response.

10. The method of claim 1, wherein transmitting the one or more ultrasound waves is performed at least in part by energizing individual transducers of a plurality of transducers at discrete times.

11. The method of claim 1, further comprising:
performing an ultrasound scan of a patient anatomy, the patient anatomy containing the location of interest;
generating an ultrasound image using the ultrasound scan;
identifying the location of interest; and
identifying one or more features in the ultrasound image which can be used to localize the location of interest.

12. A method, comprising:
using at least one of specular reflection of ultrasound waves from a location of interest or diffuse reflection of ultrasound waves from the location of interest to determine information about bone and/or other hard tissue comprising at least a portion of the location of interest.

13. The method of claim 12, wherein the information about bone and/or other hard tissue comprises the existence of a defect in the bone and/or other hard tissue.

14. The method of claim 12, further comprising:
collecting a measure of at least one of the specular reflection of ultrasound waves from the location of interest or the diffuse reflection of the ultrasound waves from the location of interest.

15. The method of claim 12, wherein using the at least one of specular reflection of ultrasound waves from the location of interest or diffuse reflection of ultrasound waves from the location of interest comprises:
comparing the specular reflection or the diffuse reflection to previously acquired data.

16. The method of claim 12, wherein using the at least one of specular reflection of ultrasound waves from the location of interest or diffuse reflection of ultrasound waves from the location of interest comprises:
comparing the specular reflection to the diffuse reflection.

17. A method for positioning an ultrasound examination device relative to a location of interest in a patient, the method comprising:
performing an ultrasound scan at a region including at least the location of interest;
determining a plurality of scan positions in which a received signal of the ultrasound scan is maximized; and
defining a scan trajectory comprising the plurality of scan positions where the received signal of the ultrasound scan is maximized.

18. The method of claim 17, further comprising storing the scan trajectory in a database.

19. The method of claim 17, further comprising using the scan trajectory to perform a subsequent ultrasound scan.

20. The method of claim 19, wherein using the scan trajectory to perform the subsequent ultrasound scan comprises:
moving an ultrasound examination device, using an automated positioning system, along the scan trajectory;
at each of the plurality of scan positions, transmitting one or more ultrasound waves to the location of interest; and
receiving one or more ultrasound waves from the location of interest, the one or more received ultrasound waves comprising reflections of at least some of the one or more transmitted ultrasound waves.
